(19) United States
(12) Patent Application Publication
(10) Pub. No.: US 2011/0243942 A1
(43) Pub. Date: Oct. 6, 2011

(54) RELAXIN-FUSION PROTEINS WITH EXTENDED IN VIVO HALF-LIVES

(75) Inventor: Tony Wang, Sunnyvale, CA (US)

(73) Assignee: ATHENA DISCOVERY, INC., Sunnyvale, CA (US)

(21) Appl. No.: 13/079,262

(22) Filed: Apr. 4, 2011

Related U.S. Application Data
(60) Provisional application No. 61/320,688, filed on Apr. 2, 2010.

(51) Int. Cl. A61K 39/395; C07K 19/00; A61K 39/44; C12N 5/10; C12N 15/03; A61P 9/00; A61P 17/02

(52) U.S. Cl. 424/134.1; 530/387.3; 536/23.4; 435/325; 435/320.1

(57) ABSTRACT

Disclosed are human relaxin-Fc fusion proteins having an increased serum half-life, polynucleotides encoding the same, and intermediates formed during the fusion protein biosynthesis. The fusion proteins may include a linker portion or other sections as well. Suitable fusion proteins are also those predicted to have the same effect as human relaxin in vivo, based, for example, on structural modeling. The fusion protein is useful in the treatment of a number of diseases and conditions, including heart disease, vascular disease, wound healing, fibrosis, fibromyalgia, and promoting angiogenesis.

### RELATED APPLICATIONS

This application claims priority to Provisional No. 61/320,688 filed Apr. 2, 2010.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Relaxin is a peptide hormone that is similar in size and shape to insulin. The active form of the encoded protein consists of an A chain and a B chain, held together by disulphide bonds, two inter-chains and one intra-chain.

Relaxin is an endocrine and autocrine/paracrine hormone which belongs to the insulin gene superfamily. In humans, there are three known non-allelic relaxin genes, relaxin-1 (RLN-I or H1), relaxin-2 (RLN-2 or H2) and relaxin-3 (RLN-3 or H3; SEQ ID NO. 1). H1 and H2 share high sequence homology. There are two alternatively spliced transcript variants encoding different isoforms described for this gene. H1 and H2 are differentially expressed in reproductive organs (see U.S. Pat. No. 5,023,321 and Garibay-Tupas et al. (2004) Molecular and Cellular Endocrinology 219:115-125) while H3 is found primarily in the brain. The evolution of the relaxin peptide family and its receptors is generally well known in the art (see Wilkinson et al. (2005) BMC Evolutionary Biology 5(14):1-17; and Wilkinson and Bathgate (2007) Chapter 1, Relaxin and Related Peptides, Landes Bioscience and Springer Science+Business Media).

Mature human relaxin is approximately 6000 daltons, is known to show a marked increase in concentration during pregnancy in many species, and is known in some species to be responsible for remodeling the reproductive tract before parturition, thus facilitating the birth process. Relaxin was discovered by F. L. Hisaw (Proc. Soc. Exo. Biol. Med. 23, 661 (1962)) and received its name from Fevold et al. (J. Am. Chem. Soc. 52, 3340 (1930)) who obtained a crude aqueous extract of this hormone from sow corpora lutea. Naturally occurring relaxin is synthesized as a single-chain 23 kDa preprorelaxin with the overall structure: signal peptide, B-chain, connecting C-chain, and A-chain. During the biosynthesis of relaxin, the signal peptide is removed as the nascent chain is moved across the endoplasmic reticulum producing the 19-kDa prorelaxin (Reddy et al., Arch. Biochem. Biophys. 294, 579, 1992). Further processing of the prorelaxin to relaxin occurs in vivo through the endoproteolytic cleavage of the C-peptide at specific pairs of basic amino acid residues located at the B/C-chain and A/C-chain junctions, after the formation of disulfide bridges between the B- and A-chains (Marriott et al. Mol. Endo. vol. 6 no. 9, 1992) in a manner analogous to insulin processing. For some characterized isoforms, the relaxin disulfide bridges occur between the cysteines at A9-B10 and A22-B22 with an intra-chain disulfide bridge within the A-chain between A8 and A13 (U.S. Pat. No. 4,656,249, issued Apr. 7, 1987).

Relaxin is found in both women and men (see Tregear et al.; Relaxin 2000, Proceedings of the Third International Conference on Relaxin & Related Peptides (22-27 Oct. 2000, Broome, Australia). In women, relaxin is produced by the corpus luteum of the ovary, the breast and, during pregnancy, also by the placenta, chorion, and decidua. In men, relaxin is produced in the testes. Relaxin levels rise after ovulation as a result of its production by the corpus luteum and its peak is reached during the first trimester, and it declines toward the end of pregnancy. In the absence of pregnancy its level declines.

In humans, relaxin plays a role in pregnancy, in enhancing sperm motility, regulating blood pressure, controlling heart rate and releasing oxytocin and vasopressin. In animals, relaxin widens the pubic bone, facilitates labor, softens the cervix (cervical ripening), and relaxes the uterine musculature. In animals, relaxin also affects collagen metabolism, inhibiting collagen synthesis and enhancing its breakdown by increasing matrix metalloproteinases. It also enhances angiogenesis and is a renal vasodilator.

Relaxin has the general properties of a growth factor and is capable of altering the nature of connective tissue and influencing smooth muscle contraction. H1 and H2 are believed to be primarily expressed in reproductive tissue while H3 is known to be primarily expressed in brain (supra). However, as discussed for example in WO 2009/140657, H2 and H3 play a major role in cardiovascular and cardiorenal function. H1 may have similar action due to its homology to H2.

Relaxin has recently shown promise in treatment of heart failure, neurodegenerative disease, hypertension, dyspnea, induction of angiogenesis, and improved wound healing (U.S. Pat. No. 5,166,191, WO 2009/140657, WO 2009/140659, WO 1993/003755, U.S. Pat. No. 6,723,702, U.S. Pat. No. 6,211,147, U.S. Pat. No. 6,780,836, and WO 2009/140433, all incorporated by reference herein). However, the utility of relaxin in all of these applications is limited to the relatively short serum half-life of relaxin, with measurements ranging from 16.6 minutes to 2 hours (Paccamonti et al. (1991), Theriogenology 35(6): 1131-1146; and Unemori et al. (1996), Journal of Clinical Investigation 98(12): 2739-2745). As a result of this short half-life, one requires large, frequent doses to achieve a noticeable effect, which in turn requires large scale production.

### SUMMARY

The inventions described and claimed herein have many attributes and embodiments including, but not limited to, those set forth or described or referenced in this Summary. It is not intended to be all-inclusive and the inventions described and claimed herein are not limited to or by the features or embodiments identified in this Summary, which is included for purposes of illustration only and not restriction.

The serum half-life of human relaxin can be extended to several days or weeks by forming a fusion protein with the immunoglobulin Fc portion, while maintaining or enhancing the biological activity, as compared with the human relaxin molecule. Similar extension of serum half-life and similar biological activity were achieved when using a human relaxin-linker-Fc fusion protein, where the linker is SEQ ID No. 2 (GGSGGSGGGGSGGGGS). Use of other linkers in the fusion protein, including linkers with GS in various proportions, are also expected to behave similarly. A number of Fc portions and fragments can also be incorporated in the fusion protein, including IgG Fc, and preferably, the IgG Fc γ4 chain. The γ4 chain is preferred over γ1 chain because the former has little or no complement activating ability.

The linker of SEQ ID No. 2 is designed to form a non-immunogenic linkage between the relaxin C terminal end and the N-terminal end of the Fc portion, as this linker is known to have such properties, as discussed in U.S. Pat. Nos. 5,908,626 and 5,723,125 (both incorporated by reference). Other linkers which include Glycine or Serine can also form such non-immunogenic linkage, as can other polymers. A G-S containing linker consists primarily of a T cell inert sequence, to reduce immunogenicity at the fusion point. If the linker was not present, the new sequence consisting of the fusion point residues (where Fc is fused to relaxin) could be a neoantigen for a human. An appropriate linker is one which allows the fusion protein to maintain its function, as determined by structural analysis.

The invention also includes a human relaxin-Fc fusion protein including an affinity tag which can be bound to aid in purification of the fusion protein, following its biosynthesis. Affinity tags are removable by chemical agents or by enzymatic means, such as proteolysis or intein splicing. Some common protein tags include chitin binding protein (CBP), maltose binding protein (MBP), and glutathione-S-transferase (GST), Isopeptag, Histidine-tag, and HA-tag. To purify using a tag: one passes tagged biosynthetic products through a column with the binding agents bound to a solid support in the column. Because the tag is designed to insert in the middle of the C-chain of pro-relaxin, the run-through portion is the right form of relaxin-Fc from which c-chain has been cleaved.

The invention also includes intermediates formed during the biosynthesis of the human relaxin-Fc fusion protein, or the human relaxin-linker-Fc fusion protein, or either product with an affinity tag, or any of the products schematically depicted in FIG. 9. During biosynthesis the human relaxin is attached to the CCA 3′ end of a tRNA by covalent linking. This reaction follows reaction of the C terminal amino acid of the N-terminal portion of the fusion protein (either relaxin or Fc, depending on the final fusion protein structure) with ATP to yield a protein-acyl-AMP intermediate product, which in turn reacts with tRNA to form an ester bond, thus forming a protein-acyl-tRNA intermediate. This intermediate is then a substrate for a ribosome, which catalyzes the attack of the amino group of the protein chain on the ester bond. The amino group could be on the N-terminal end of a linker or the N-terminal end of an immunoglobulin Fc portion. Or in making other constructs described herein, for example, where the N-terminal end of human relaxin is conjugated to the C-terminal end of an immunoglobulin Fc portion, an analogous intermediate would be formed, i.e., an immunoglobulin Fc portion-acyl-tRNA. Other intermediates are formed when the amino group attacks the ester bond, and these are also intermediates of the invention.

The longer half-life of the Fc fusion protein may be because of a site on Fc between the CH2 and CH3 domains, which mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766, incorporated by reference). This process, coupled with preclusion of kidney filtration due to the large size of the full length molecule, may result in the favorable antibody serum half-lives observed, ranging from one to three weeks.

Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the function of this region on Fc is useful for both the clinical properties of antibodies and their purification. Available structures of the rat Fc/FcRn complex (Martin et al., 2001, Mol Cell 7:867-877, incorporated by reference), and of the complexes of Fc with proteins A and G (Deisenhofer, 1981, Biochemistry 20:2361-2370; Sauer-Eriksson et al., 1995, Structure 3:265-278; Tashiro et al., 1995, Curr Opin Struct Biol 5:471-481, incorporated by reference) provide insight into the interaction of Fc with these proteins.

Non-limiting examples of conditions for which the fusion protein can be administered to ameliorate include orthodontics-related conditions, fibromyalgia, fibrosis and heart failure or other related or unrelated heart conditions, including acute decompensated heart failure and classes I, II, III, and IV heart failure; sinus bradycardia; neurodegenerative disease; wounds to tissues, including skin; dyspnea; ischemic wounds and other ischemic conditions; infection; hypertension; renal dysfunction; pulmonary arterial hypertension; inflammation; and fibrosis. Other conditions and applications in which the fusion protein of the present invention can find use include, but are not limited to, promoting angiogenesis, promoting cardiac or vascular function, including increasing the force rate of atrial contraction, increasing cardiac output, stimulating cardiac inotropy, stimulating cardiac chronotropy, restoring cardiac function following heart failure, increasing heart rate (such as to a normal level), reducing use of heart failure medications (taken concurrently or non-concurrently), increasing cardiac index, reducing hospital stay duration associated with heart failure, promoting angiogenesis, inducing secretion of vascular endothelial growth factor (VEGF), reducing hypertension, increasing vasodilation, increasing a parameter associated with a renal function, increasing the production of an angiogenic cytokine, increasing nitric oxide production in a cell (including a cell of a blood vessel), increasing endothelin type B receptor activation in a cell of a blood vessel, increasing arterial compliance, and increasing intrauterine fetal growth rate. Other conditions are described further below.

Other uses for the fusion proteins are promoting wound healing, wherein the term “wound” includes an injury to any tissue, including, for example, acute, delayed or difficult to heal wounds, and chronic wounds. Examples of wounds may include both open and closed wounds. Wounds include, for example, burns, incisions, excisions, lacerations, abrasions, puncture on penetrating wounds, surgical wounds, contusions, hematoma, crushing injuries and ulcers. Also included are wounds that do not heal at expected rates. The term “wound” may also include for example, injuries to the skin and subcutaneous tissue initiated in different ways (e.g., pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

### BRIEF DESCRIPTION OF THE DRAWINGS

FIG. 1A illustrates an expression vector containing a polynucleotide encoding a human relaxin-Fc fusion protein.

FIG. 1B illustrates other polynucleotide inserts encoding other fusion proteins than those in FIG. 1A, including ones with linkers where the linker has amino acid content (G4S)3 and affinity tags on C chain.

FIG. 2A illustrates an expression vector containing a polynucleotide encoding a human relaxin-Fc fusion protein including an IRES (internal ribosome entry) region.

FIG. 2B illustrates other polynucleotide inserts encoding other fusion proteins than those in FIG. 1A, including ones with linkers and Fc mutant regions, where the linker has amino acid content (G4S)3.

FIG. 3A shows the amino acid sequence of human H2 relaxin (Relaxin(H2)), beginning with the signal peptide (italics), followed by the B chain (bold), C chain, and A chain (bold, itallics).

FIG. 3B shows an exemplary Fc-γ1 fragment sequence.

FIG. 3C shows an exemplary Fc-γ4 fragment sequence.

FIG. 4A shows the amino acid sequence of a His-tagged human H2 relaxin fusion protein.

FIG. 4B shows the amino acid sequence of a human H2 relaxin-Fc-γ1 fusion protein.

FIG. 4C shows the amino acid sequence of a human H2 relaxin-Fc-γ4 fusion protein.

FIG. 5A shows the amino acid sequence of a human H2 relaxin-linker-Fc-γ1 fusion protein.

FIG. 5B shows the amino acid sequence of a human H2 relaxin-linker-Fc-γ1 fusion protein, wherein the Fc region is not wild-type.

FIG. 5C shows the amino acid sequence of a human H2 relaxin-linker-Fc-γ4 fusion protein.

FIG. 6A shows the predicted structure of a human H2 relaxin-Fc fusion protein.

FIG. 6B shows the predicted structure of a human H2 relaxin-linker-Fc fusion protein.

FIG. 7A shows predicted structure of a Fc-Relaxin fusion protein.

FIG. 7B shows predicted structure of a Fc-linker-Relaxin fusion protein.

FIG. 7C shows predicted structure of a Relaxin-Fc-Relaxin fusion protein.

FIG. 8A shows the predicted structure of a pre-Relaxin peptide.

FIG. 8B shows the predicted structure of a mature Relaxin peptide.

FIG. 9 shows the structure of various Relaxin-Fc fusion proteins, wherein: Fc fragment is the human antibody IgG heavy chain γ4 or γ1; Relaxin can be fused to Fc at the N-terminus or C-terminus thereof; C-chain of Relaxin can be the full length or shortened length, or mutated, or including an affinity tag, for purification purposes, which can be a His Tag, an HA Tag, or others; Linker can be (G4S)3, or GGSGGSGGGGSGGGGS, or others.

FIG. 10 shows SDS-PAGE separation results with Relaxin-(L)-Fc and Relaxin-Fc with and without C-chain and with and without affinity tag purification.

FIG. 11 shows Western Blot results for the Relaxin molecule AD2 of FIG. 9, and for an Fc fragment.

FIG. 12 shows the in vitro biological activity of Relaxin-Fc vs. Relaxin-(L)-Fc, as measured by intracellular cAMP release.

FIG. 13 shows the PKs, as measured in an animal model, of native Relaxin, Relaxin-Fc and Relaxin-(L)-Fc.

FIG. 14 shows that Relaxin-Fc inhibits TGF-β1 induced ET-1 production in HLF cells.

FIG. 15 shows that Relaxin-Fc reduces bleomycin-induced lung fibrosis in a mouse model.

FIG. 16 shows that Relaxin-Fc increases urine flow rate in a rat model.

FIG. 17A shows the crystal structure of Relaxin-2.

FIG. 17B shows the predicted crystal structure of Relaxin-Fc.

FIG. 17C shows the crystal structure of Relaxin-2 from a different angle than in FIG. 17A.

FIG. 17D shows the predicted crystal structure of Relaxin-Linker-Fc, where the linkers are: (Gly4Ser)3, (Ser-Gly-(Ser-Ser-Ser-Ser-Gly)2-Ser), (Gly-Gly-Ser-Gly)n (n=1-5), or (Ser-Gly-(Ser-Ser-Ser-Ser-Gly)2-Ser-).

### SEQUENCE LISTING INDEX

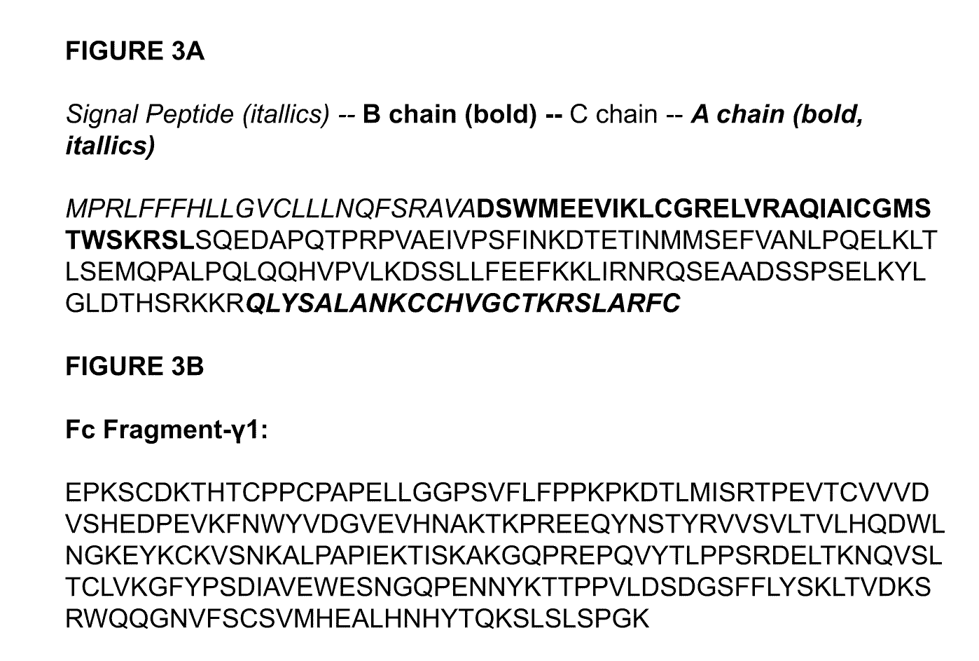

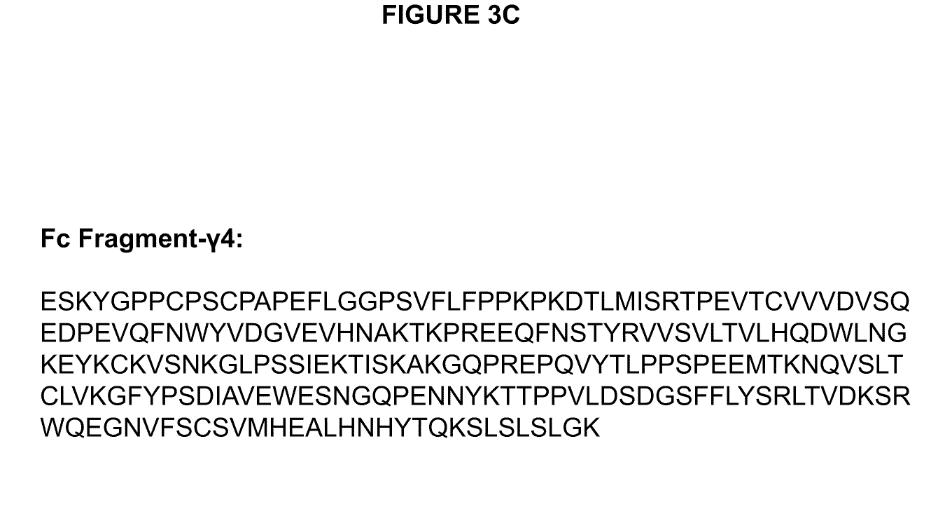

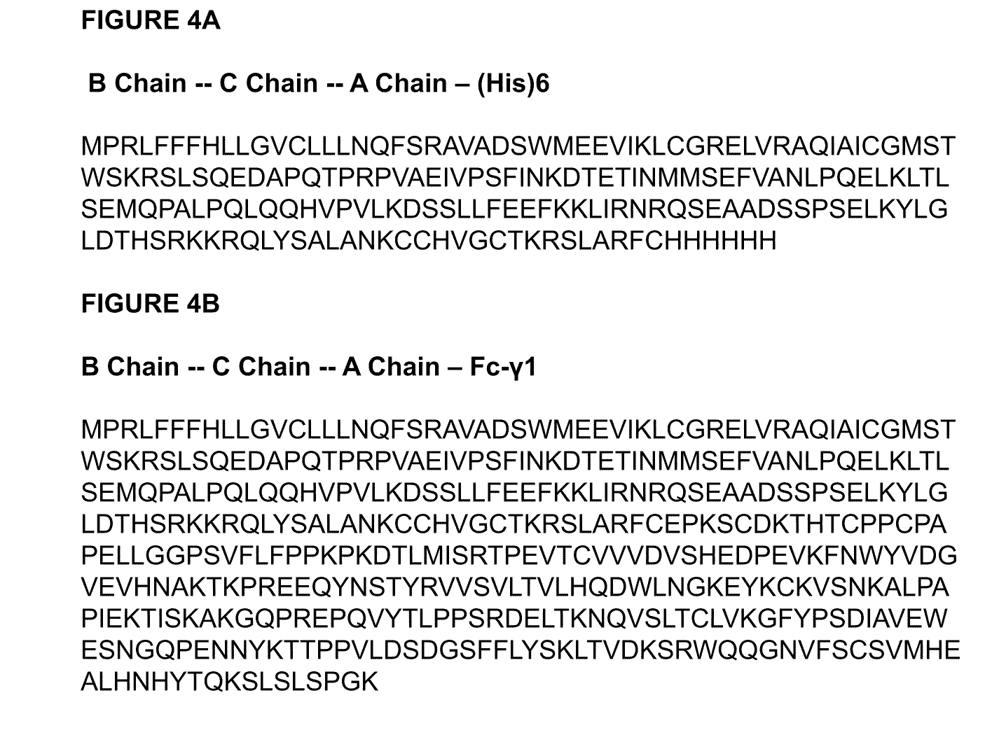

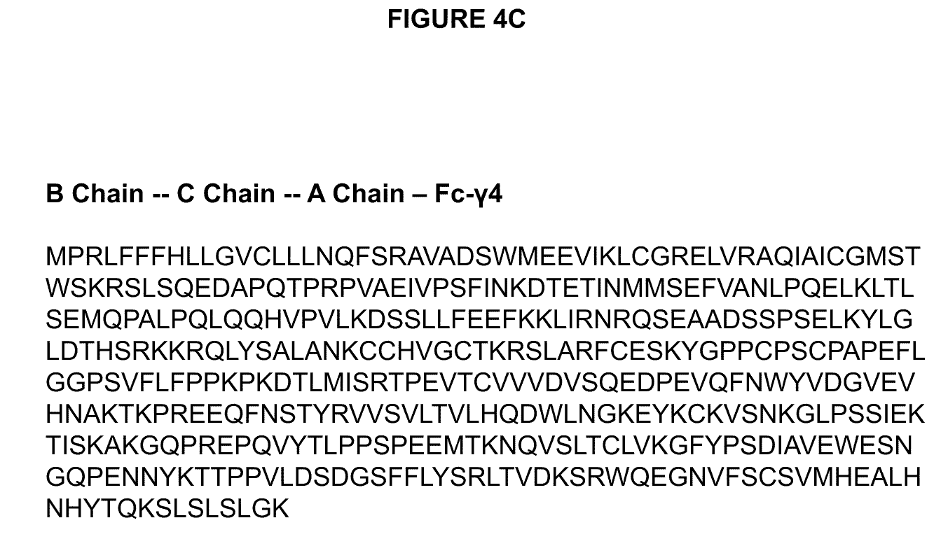

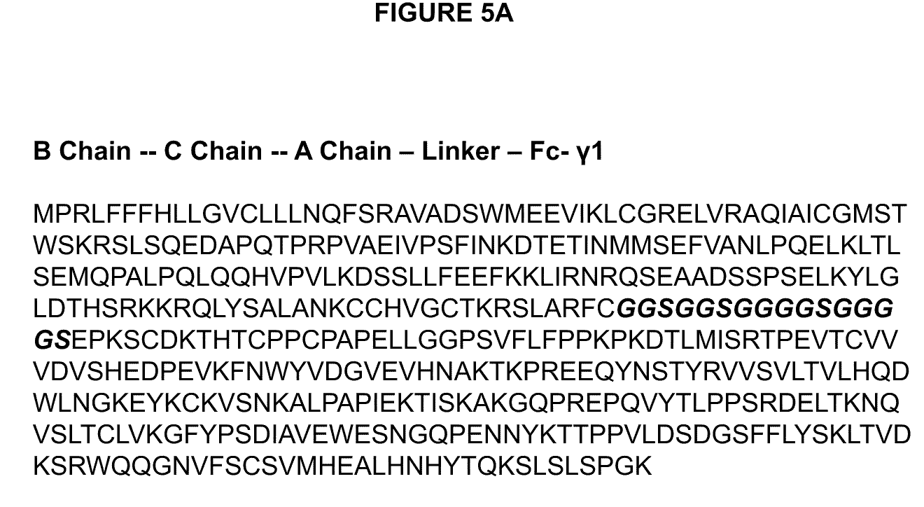

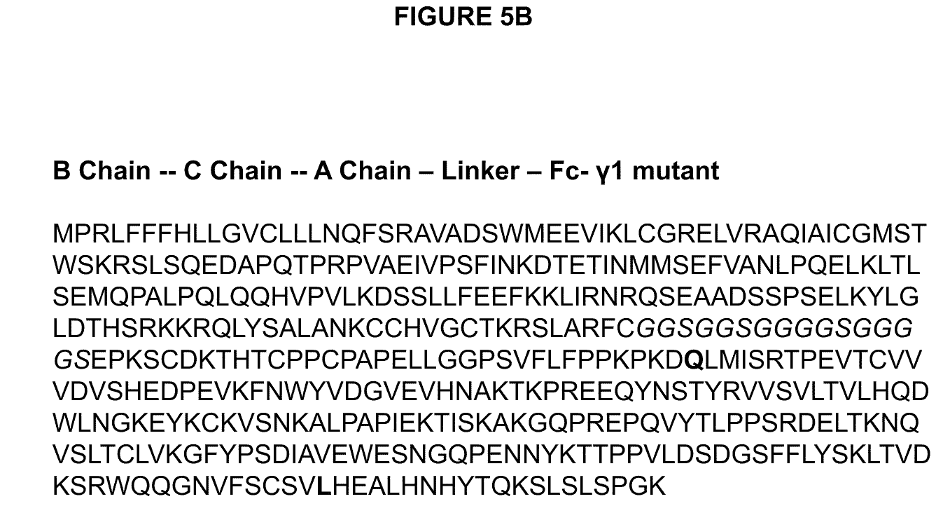

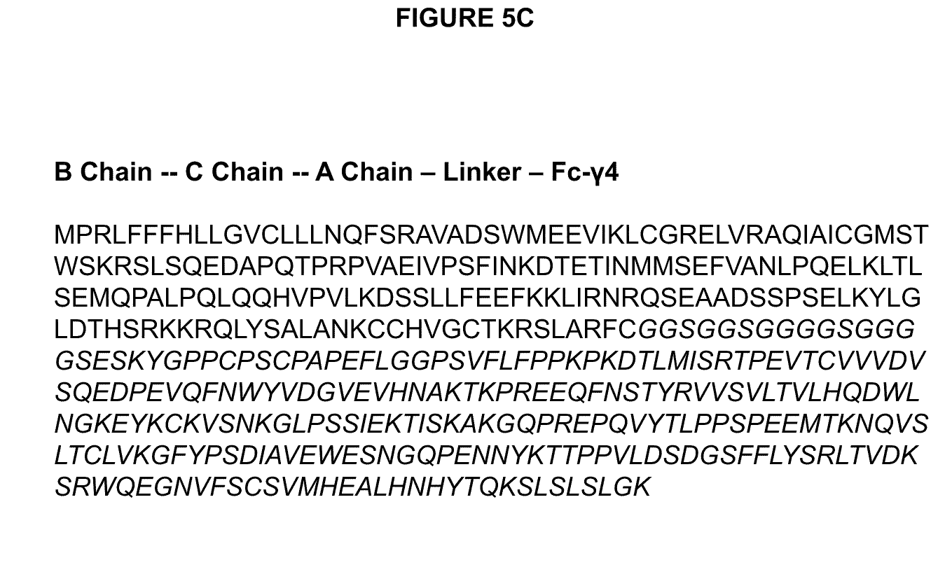

SEQ ID No. 1 is the DNA sequence of human Relaxin-3.
SEQ ID No. 2 is the amino acid sequence of a linker of the invention.
SEQ ID No. 3 (shown in FIG. 3A) is the amino acid sequence of human relaxin-2 (RLN-2 or H2) showing signal peptide, A, B and C chains.
SEQ ID No. 4 (shown in FIG. 3B) is the amino acid sequence of a human Fc-γ1.
SEQ ID No. 5 (shown in FIG. 3C) is the amino acid sequence of a human Fc-γ4
SEQ ID No. 6 (shown in FIG. 4A) is the amino acid sequence of a human relaxin-2 with a histidine affinity tag at its C-terminal end.
SEQ ID No. 7 is the DNA sequence of the human relaxin-2 with a histidine affinity tag at its C-terminal end of SEQ ID No. 6.
SEQ ID No. 8 (shown in FIG. 4B) is the amino acid sequence of a human relaxin-2-Fc-γ1 fusion protein.
SEQ ID No. 9 is the DNA sequence of human relaxin-2-Fc-γ1 fusion protein of SEQ ID No. 8.
SEQ ID No. 10 (shown in FIG. 4C) is the amino acid sequence of a human relaxin-2-Fc-γ4 fusion protein
SEQ ID No. 11 (shown in FIG. 5A) is the amino acid sequence of a human relaxin-2-Linker-Fc-γ1 fusion protein.
SEQ ID No. 12 is the DNA sequence of human relaxin-2-Linker-Fc fusion protein of SEQ ID No. 11.
SEQ ID No. 13 (shown in FIG. 5B) is the amino acid sequence of a human relaxin-2-Linker-Fc-γ1 fusion protein, where the Fc is a mutant form.
SEQ ID No. 14 is the DNA sequence of the human relaxin-2-Linker-Fc mutant fusion protein of SEQ ID No. 13.
SEQ ID No. 15 (shown in FIG. 5C) is the amino acid sequence of a human relaxin-2-Linker-Fc-γ4 fusion protein.

### DEFINITIONS

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may include non nucleotide components. A polynucleotide may be further modified after polymerization, such as by conjugation with a labeling component.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The terms “biologically active” and “bioactive,” as used herein, indicate that a composition or compound itself has a biological effect, or that it modifies, causes, promotes, enhances, blocks, or reduces a biological effect, or which limits the production or activity of, reacts with and/or binds to a second molecule that has a biological effect. The second molecule can be, but need not be, endogenous. A “biological effect” can be but is not limited to one that stimulates or causes an immunoreactive response; one that impacts a biological process in a cell, tissue or organism (e.g., in an animal); one that generates or causes to be generated a detectable signal; and the like.

Biologically active compositions, complexes or compounds may be used in investigative, therapeutic, prophylactic, and/or diagnostic methods and compositions. Biologically active compositions, complexes or compounds act to cause or stimulate a desired effect upon a cell, tissue, organ or organism (e.g., an animal). Non-limiting examples of desired effects include modulating, inhibiting or enhancing gene expression in a cell, tissue, organ, or organism; preventing, treating or curing a disease or condition in an animal suffering therefrom; and stimulating a prophylactic immunoreactive response in an animal.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

The term “formulation” includes delivery forms and formulations for the fusion proteins herein which deliver an effective amount of the fusions proteins to a subject. Preferred formulations include, for example, a pharmaceutical compositions which are formulated as an injection, tablet, capsule, sublingual, topical, transdermal or other formulation.

### DETAILED DESCRIPTION

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Fusion proteins of the invention comprises A and B chains of a human relaxin (Relaxin-1, Relaxin-2 or Relaxin-3) and at least a portion of a constant immunoglobulin domain such that said fusion protein exhibits a longer serum half-life while maintaining therapeutic effect, relative to the corresponding human relaxin. In general, the term “fusion protein” refers to a protein that is a conjugate of domains obtained from more than one protein or polypeptide. The terms “fusion protein,” “fusion peptide,” “fusion polypeptide,” and “chimeric peptide” are used interchangably. Domains can comprise full-length proteins, fragments of proteins, proteins of modified amino acid sequence, proteins incorporating modified amino acids, amino acid sequences derived from an organism, artificial amino acid sequences, proteins joined by disulfide bonds, linkers, tags, or combinations thereof. The conjugates can be prepared by linking the domains by chemical conjugation, recombinant DNA technology, or combinations of recombinant expression and chemical conjugation. Where recombinant DNA technology is used in the generation of a fusion protein, the fusion protein is generally expressed such that each domain is part of a single amino acid sequence. A fusion protein can also comprise a processed protein, the domains of which were part of a single amino acid sequence prior to processing.

### Relaxin

The term “human relaxin” or “relaxin” or “RLX” or includes any human relaxin from recombinant or native sources as well as human relaxin variants, such as amino acid sequence variants. A human relaxin of the present invention can comprise other insertions, substitutions, or deletions of one or more amino acid residues, glycosylation variants, unglycosylated human relaxin, covalently modified derivatives of human relaxin, human preprorelaxin, and human prorelaxin. Through the use of recombinant DNA technology, relaxin variants can be prepared by altering the underlying DNA. All such variations or alterations in the structure of the relaxin molecule resulting in variants are included within the scope of this invention. In some embodiments, the human relaxin is H1, H2, or H3 relaxin, or isoforms, splice variants, combinations, and/or other variants thereof, such as those described in U.S. Pat. No. 4,758,516, U.S. Pat. No. 4,871,670, U.S. Pat. No. 5,811,395, U.S. Pat. No. 5,759,807, U.S. Pat. No. 5,145,962, U.S. Pat. No. 5,179,195, US 2005/0026822, and WO 2009/055854. Human relaxin further encompases human H1 preprorelaxin, prorelaxin, and relaxin; H2 preprorelaxin, prorelaxin, and relaxin; and H3 preprorelaxin, prorelaxin, and relaxin. Human relaxin further includes biologically active (also referred to herein as “pharmaceutically active”) relaxin from recombinant, synthetic or native sources as well as relaxin variants, such as amino acid sequence variants. As such, the terms “human relaxin” or “relaxin” or “RLX” contemplate synthetic human relaxin and recombinant human relaxin, including synthetic H1, H2 and H3 human relaxin, recombinant H1, H2 and H3 human relaxin, and combinations thereof.

Human relaxin can comprise amino acid sequence elements from different human relaxins, including but not limited to signal peptides, A chains, B chains, C chains, or portions thereof derived from different human relaxins or variants thereof. In some embodiments, the polynucleotide sequence encoding the human relaxin of the human relaxin fusion protein is able to hybridize with a polynucleotide encoding human H1, H2, and/or H3 relaxin. In still other embodiments, the polynucleotide sequence encoding the human relaxin of the human relaxin fusion protein has at least about 85% or more sequence identity to human H1, H2, and/or H3 relaxin. In some embodiments, the signal peptide is derived from another protein, an artificial signal peptide sequence, or combinations or variants thereof. In some embodiments, human relaxin is characterized by an ability to bind a relaxin receptor. Relaxin receptors include any receptor to which human H1, H2, and/or H3 relaxin can bind, including but not limited to RXFP1, RXFP2, RXFP3, RXFP4, FSHR (LGR1), LHCGR (LGR2), TSHR (LGR3), LGR4, LGR5, LGR6, LGR7 (RXFP1), and LGR8 (RXFP2).

Also encompassed by the terms “human relaxin” or “relaxin” or “RLX” is relaxin modified to increase in vivo half life, e.g., PEGylated relaxin (i.e., relaxin conjugated to a polyethylene glycol), relaxin which is modified such that amino acids in relaxin that are subject to cleavage by degrading enzymes are altered, deleted or modified, and the like.

Human relaxin also encompasses relaxin comprising A and B chains having N- and/or C-terminal truncations. In one embodiment, in H2 relaxin, the A chain can be varied from A(1-24) to A(10-24) and B chain from B(1-33) to B(10-22); and in H1 relaxin, the A chain can be varied from A(1-24) to A(10-24) and B chain from B(1-32) to B(10-22). Also encompassed in the term is a relaxin analog having an amino acid sequence which differs from a wild-type (e.g., naturally-occurring) sequence, including, but not limited to, relaxin analogs disclosed in U.S. Pat. No. 5,811,395. Possible modifications to relaxin amino acid residues include the acetylation, formylation or similar protection of free amino groups, including the N-terminal groups, amidation of C-terminal groups, or the formation of esters of hydroxyl or carboxylic groups, e.g., modification of the tryptophan (Trp) residue at B3 by addition of a formyl group. The formyl group is a typical example of a readily-removable protecting group. Other possible modifications include replacement of one or more of the natural amino-acids in the B and/or A chains with a different amino acid (including the D-form of a natural amino-acid), including, but not limited to, replacement of the Met moiety at B25 with norleucine (NIe), valine (Val), alanine (Ala), glycine (GIy), serine (Ser), or homoserine (HomoSer). Other possible modifications include the deletion of a natural amino acid from the chain or the addition of one or more extra amino acids to the chain. Additional modifications include amino acid substitutions at the B/C and C/A junctions of prorelaxin, which modifications facilitate cleavage of the C chain from prorelaxin; and variant relaxin comprising a non-naturally occurring C peptide, e.g., as described in U.S. Pat. No. 5,759,807.

“Human relaxin” or “relaxin” or “RLX” also includes fusion polypeptides comprising relaxin and a heterologous polypeptide. A heterologous polypeptide (e.g., a non-relaxin polypeptide) fusion partner may be C-terminal or N-terminal to the relaxin portion of the fusion protein. Heterologous polypeptides include immunologically detectable polypeptides (e.g., “epitope tags”); polypeptides capable of generating a detectable signal (e.g., green fluorescent protein, enzymes such as alkaline phosphatase, and others known in the art); therapeutic polypeptides, including, but not limited to, cytokines, chemokines, and growth factors; and constant immunoglobulin domain polypeptides, and affinity tags. Preferably, any modification of relaxin amino acid sequence or structure is one that does not increase its immunogenicity in the individual being treated with the relaxin variant. Those variants of relaxin having the described functional activity can be readily identified using in vitro and in vivo assays known in the art.

In some embodiments, the A and B chains are derived from the same human relaxin. In other embodiments, the A and B chains are derived from different human relaxins. In still other embodiments, the A and/or B chain comprise sequences from two or more human relaxins or variants thereof. In some embodiments the human relaxin A chain has at least 85% or more amino acid sequence homology to the A chain of human H1, H2, or H3 relaxin. In some embodiments the human relaxin B chain has at least 85% or more amino acid sequence homology to the B chain of human H1, H2, or H3 relaxin. In some embodiments, the A and B chains of human relaxin are expressed as part of a single transcript. In other embodiments, the A and B chains of human relaxin are expressed as parts of separate transcripts.

### Immunoglobulin Domain

In some embodiments, the fusion protein comprises a constant immunoglobulin domain, such as a constant heavy immunoglobulin domain, a constant light immunoglobulin domain, or portions, combinations, or variants thereof. The constant immunoglobulin domain can be derived from the constant region of any immunoglobulin, including but not limited to IgA, IgD, IgE, IgG, IgM, and combinations and/or variants thereof. In some embodiments, the source immunoglobulin is an IgG. IgG can be further divided into IgG1, IgG2, IgG3 and IgG4 subclasses, and the present invention includes domains derived from combinations and hybrids thereof. Immunoglobulin domains can be derived from the immunoglobulins of any Gnathostomata, including but not limited to mammals, such as humans. In some embodiments, the constant immunoglobulin domain comprises an Fc fragment. The term “Fc fragment” or “Fc” as used herein, refers to a protein that contains the heavy-chain constant region 2 (CH2) and the heavy-chain constant region 3 (CH3) of an immunoglobulin, and not the variable regions of the heavy and light chains. It may further include the hinge region of the heavy-chain constant region. Also, the immunoglobulin Fc fragment of the present invention may contain a portion or all of the heavy-chain constant region 1 (CH1), heavy-chain constant region 4 (CH4) and/or the light-chain constant region 1 (CL1), except for the variable regions of the heavy and light chains. Also, the Fc fragment may be a fragment having a deletion in a relatively long portion of the amino acid sequence of CH2 and/or CH3. That is, the Fc fragment of the present invention can comprise 1) a CH1 domain, a CH2 domain, a CH3 domain and a CH4 domain, 2) a CH1 domain and a CH2 domain, 3) a CH1 domain and a CH3 domain, 4) a CH2 domain and a CH3 domain, 5) a combination of one or more domains and an immunoglobulin hinge region (or a portion of the hinge region), or 6) a dimer-of each domain of the heavy-chain constant regions and the light-chain constant region. In some embodiments, the Fc fragment of the human relaxin fusion protein comprises combinations of CH1, CH2, CH3, CH4, and/or hinge regions the same or different immunoglobulins from the same or different Gnathostomata, including but not limited to humans, cows, goats, swine, mice, rabbits, hamsters, rats and guinea pigs. In other embodiments, sub-sequences within CH1, CH2, CH3, CH4, and/or hinge regions of an Fc fragment are derived from the same or different immunoglobulins from the same or different Gnathostomata, including but not limited to humans, cows, goats, swine, mice, rabbits, hamsters, rats and guinea pigs. In some embodiments, the constant immunoglobulin domain comprises an Fc region of a heavy chain IgG immunoglobulin, including preferably the gamma-4 region, as the gamma-1 region can activate complement.

The Fc fragments of the present invention include a native amino acid sequence and sequence derivatives (mutants) thereof. An amino acid sequence derivative is a sequence that is different from the native amino acid sequence due to a deletion, an insertion, a non-conservative or conservative substitution or combinations thereof of one or more amino acid residues. In some embodiments, the Fc fragment comprises amino acid sequences with at least some substantial homology to the Fc region of an immunoglobulin from any Gnathostomata, including but not limited to humans, cows, goats, swine, mice, rabbits, hamsters, rats and guinea pigs. For example, in an IgG Fc, amino acid residues known to be important in binding, at positions 214 to 238, 297 to 299, 318 to 322, or 327 to 331, may be used as a suitable target for modification. Also, other various derivatives are possible, including one in which a region capable of forming a disulfide bond is deleted, or certain amino acid residues are eliminated at the N-terminal end of a native Fc form or a methionine residue is added thereto. Further, to remove effector functions, a deletion may occur in a complement-binding site, such as a C1q-binding site and an ADCC (antibody dependent cellular cytotoxicity) site. Examples of such deletions are described, for example, in U.S. Pat. No. 7,030,226. In addition, the Fc fragment, if desired, may be modified by phosphorylation, sulfation, acrylation, glycosylation, methylation, farnesylation, acetylation, amidation, and the like.

Other Fc modifications that are considered include those that increase functions, such as altered binding to Fc receptors and/or altered serum half-life. Fc fragment variants can include those with increased or decreased binding affinity for Fc receptors relative to unmodified Fc fragments, and can also include Fc fragment variants with increased or decreased serum half-lives. Examples of Fc variants having altered binding affinities and serum half-lives are described in US 2005/0226864.

In addition, Fc fragments can be obtained from native forms isolated from humans and other animals including cows, goats, swine, mice, rabbits, hamsters, rats and guinea pigs, or may be recombinants or derivatives thereof, obtained from transformed, transfected, or transgenic animals, animal cells, or microorganisms. Fc fragments can be obtained from a native immunoglobulin by isolating whole immunoglobulins from human or animal organisms and treating them with a proteolytic enzyme. Papain digests the native immunoglobulin into Fab and Fc fragments, and pepsin treatment results in the production of pFc and F (ab′) 2 fragments. These fragments may be subjected, for example, to size exclusion chromatography. Alternatively, Fc fragments can be obtained by expression in transformed, transfected, or transgenic cells or organisms, including as part of a fusion protein.

In addition, the immunoglobulin Fc fragment of the present invention can be in the form of having native sugar chains, increased sugar chains compared to a native form or decreased sugar chains compared to the native form, or may be in a deglycosylated form. The increase, decrease, or removal of the immunoglobulin Fc sugar chains may be achieved by methods common in the art, such as a chemical method, an enzymatic method, and/or a genetic engineering method using a microorganism. The removal of sugar chains from an Fc fragment results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. In this regard, an immunoglobulin Fc fragment in a deglycosylated or aglycosylated form.

An antibody dependent cellular cytotoxicity (ADCC) assay can be employed to screen the fusion proteins of the present invention having mutant ADCC sites. ADCC assays can be performed in vitro or in vivo. To assess ADCC activity of a polypeptide variant, an in vitro ADCC assay can be performed using varying effector to target ratios. An exemplary ADCC assay could use a target cell line expressing a relaxin receptor. Effector cells may be obtained from a healthy donor (e.g. on the day of the experiment) and PBMC purified using Histopaque (Sigma). Target cells are then preincubated with a human relaxin fusion protein at, for example, 1-10 μg/mL for about 30 minutes prior to mixing with effector cells at effector:target ratios of, for example, 40:1, 20:1 and 10:1. ADCC activity may then be measured calorimetrically using a Cytotoxicity Detection Kit (Roche Molecular Biochemicals) for the quantitation of cell death and lysis based upon the measurement of lactate dehydrogenase (LDH) activity released from the cytosol of damaged cells into the supernatant. ADCC activity can also be measured, for Chromium 51 loaded target cell assays, by measuring the resulting Chromium 51 released. Antibody independent cellular cytoxicity can be determined by measuring the LDH activity from target and effector cells in the absence of antibody. Total release may be measured following the addition of 1% triton X-100 to the mixture of target and effector cells. Incubation of the target and effector cells can be performed for an optimized period of time (4-18 hours) at 37° C. in 5.0% CO₂ and then be followed by centrifugation of the assay plates. The supernatants can then be transferred to 96-well plates and incubated with LDH detection reagent for 30 minutes at 25° C. The sample absorbance can then be measured at 490 nm using a microplate reader. The percent cytotoxicity can then be calculated using the following equation: % cytotoxicity=experimental value−low control/high control−low control×100%. The percent cytoxicity of human relaxin fusion protein with altered ADCC activity can then be compared directly with equal amount of human relaxin fusion protein with unmodified ADCC activity to provide a measurement of relative change in ADCC activity. Many variations of this assay are known in the art (See, e.g., Zuckerman et al., CRC Crit. Rev Microbiol 1978; 7(1):1-26, herein incorporated by reference). Useful effector cells for such assays includes, but are not limited to, natural killer (NK) cells, macrophages, and other peripheral blood mononuclear cells (PBMC). Alternatively, or additionally, ADCC activity of the human relaxin fusion proteins of the present invention may be assessed in vivo, e.g., in a animal model such as that disclosed in Clynes et al. PNAS (USA) 95:652-656 (1998), herein incorporated by reference).

### Serum Half Life

In some embodiments, the human relaxin fusion protein comprising at least a portion of a constant immunoglobulin domain exhibits a longer serum half-life relative to the corresponding human relaxin that lacks said constant immunoglobulin domain. Serum half-life can refer to the time it takes for a substance to lose half of its pharmacologic, physiologic, or radiologic activity following introduction of an amount of the substance into the serum of an organism. Serum half-life can also refer to the time it takes for a substance to be reduced to half of a starting amount introduced into the serum of an organism, following such introduction. In some embodiments, serum half-life is increased substantially, e.g., from minutes to several days. Biological stability (or serum half-life) can be measured by a variety of in vitro or in vivo means. For example, differences in half-life can be compared by using a radiolabeled version of each protein to be compared and measuring levels of serum radioactivity as a function of time in the same or different organism. Alternatively, serum half-life can be compared by assaying the levels exogenous human relaxin present in serum using ELISA as a function of time in the same or different organism. Assay methods for measuring in vivo pharmacokinetic parameters (e.g. in vivo mean elimination half-life) are described in U.S. Pat. No. 7,217,797, as well as alterations to the immunoglobulin Fc heavy chain, which alter its binding to the FcRn receptor.

### Domain Fusion and Order

In some embodiments, the constant immunoglobulin domain is joined to the human relaxin B chain of the fusion protein. In other embodiments, the constant immunoglobulin domain is joined to the human relaxin A chain of the fusion protein. In still other embodiments, a constant immunoglobulin domain is joined to both the human relaxin A chain and human relaxin B chain of the fusion protein. Joining can be achieved by any method known in the art, including by not limited to chemical conjugation, recombinant DNA technology, or combinations of recombinant expression and chemical conjugation. In some embodiments, the constant immunoglobulin domain is joined to the A chain and/or the B chain by an intervening amino acid sequence, or linker. In some embodiments, the linker can be virtually any number of amino acids in length. A linker can be derived from the protein of an organism, an artificially designed amino acid sequence, a random amino acid sequence, or variants, portions, or combinations thereof. Examples of linkers are described in U.S. Pat. No. 5,908,626 and by Kuttner et al. (BioTechniques 36: 864-870, 2004).

In some embodiments, the domains of the human relaxin fusion protein are arranged in a specific order. Domain order in a polypeptide can be expressed with respect to the amino-terminus (N-terminus) and carboxy-terminus (C-terminus) of the fusion protein as a whole, domains thereof, and/or individual amino acids thereof. In general, the order of domains in a polypeptide refers to those domains that are part of a single polypeptide chain, and/or were a part of a single polypeptide chain. This includes polypeptides having multiple domains translated in a particular order as a single chain that are subsequently processed into two or more separate polypeptide chains, each resulting chain preserving the N-terminus to C-terminus order of its components, but being separated from the other resulting chains from the original polypeptide. In some embodiments, each domain appearing in a specified order of domains is immediately disposed adjacent to the domain that precedes it and/or the domain the follows it in the described order of domains. For example, the C-terminal amino acid of one domain can be immediately followed by the N-terminal amino acid of the next domain in a given order of domains of a polypeptide. In other embodiments, one or more pairs of adjacent domains in a described order of domains of a polypeptide can be separated by one or more amino acids that are not part of either domain of the pair. In some embodiments, any number of intervening amino acids, polypeptide domains, and/or polypeptides may separate the domains specified in an order of domains, so long as the specified order is maintained. In some embodiments, the human relaxin fusion protein comprises, from the N-terminus to the C-terminus, the B chain, the A chain, and the constant immunoglobulin domain. In other embodiments, the human relaxin fusion protein comprises, from the N-terminus to the C-terminus, the constant immunoglobulin domain, the B chain, and the A chain.

In one embodiment, the fusion protein further comprises a C chain of a human relaxin. In some embodiments, the C chain is derived from the same human relaxin as the A chain and/or the B chain. In other embodiments, the C chain is derived from a human relaxin other than those from which the A and B chains are derived. In some embodiments, the C chain is modified by insertion, deletion, and/or substitution of the amino acid sequence and/or nucleotide sequence. In some embodiments, the C chain is a non-naturally occurring C chain, such as is described in U.S. Pat. No. 5,759,807. In still other embodiments, the C chain comprises sequences from two or more human relaxins or variants thereof. In some embodiments, the human relaxin C chain has at least substantial amino acid sequence homology to the C chain of human H1, H2, or H3 relaxin. In one embodiment, the human relaxin fusion protein comprises, from the N-terminus to the C-terminus, the B chain, a C chain of a human relaxin, the A chain, and the constant immunoglobulin domain. In another embodiment, the human relaxin fusion protein comprises, from N-terminus to C-terminus, the constant immunoglobulin domain, the B chain, a C chain of a human relaxin, and the A chain. In some embodiments, the C chain is removed from the fusion protein in a processing step. The processing step can take place inside or outside a cell.

### Receptor Binding

In one embodiment, the fusion protein competes with human relaxin for binding of a human relaxin receptor. Relaxin receptors include any receptor to which human H1, H2, and/or H3 relaxin can bind, including but not limited to RXFP1, RXFP2, RXFP3, RXFP4, FSHR (LGR1), LHCGR (LGR2), TSHR (LGR3), LGR4, LGR5, LGR6, LGR7 (RXFP1), and LGR8 (RXFP2). Competition can be assessed using standard competitive binding assays. For example, a fixed amount of unlabeled receptor can be combined with a fixed amount of labeled (e.g. radiolabeled) human relaxin and increasing amounts of unlabeled fusion protein. Competition with the labeled human relaxin is indicated by a decrease in bound label, which can be assessed by electrophoretic mobility shift assay. Efficiency of competition depends on the binding affinity of the fusion protein for the relaxin receptor. In some embodiments, the binding affinity of the fusion protein for a human relaxin receptor is less or more that of the same protein lacking the constant immunoglobulin domain. In some embodiments, the binding affinity of the fusion protein is less or more that of human H1, H2, and/or H3 relaxin. Alternatively, binding affinity can be expressed in terms of a dissociation constant (Kd), the concentration at which a binding site (e.g. a receptor) is half occupied (or the concentration of binding partner at which half of a fixed number of binding sites are occupied). In some embodiments, the Kd of the interaction between the fusion protein and a human relaxin receptor is at least about 10⁻⁶ M, 10⁻⁷ M, 10⁻⁸ M, 10⁻⁹ M, 10⁻¹⁰ M, or 10⁻¹¹ M. Standard methods for determining dissociation constants are known in the art, and can include measurements of bound fusion protein for increasing amounts of labeled fusion protein in the presence of a fixed amount of receptor.

### Cloning and Expression Vectors

In one embodiment, the invention provides recombinant polynucleotides encoding the fusion proteins. The polynucleotides of the invention can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention. The polynucleotides embodied in this invention can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service. Polynucleotides comprising a desired sequence can be inserted into a suitable vector which in turn can be introduced into a suitable host cell for replication and amplification. Accordingly, the invention encompasses a variety of vectors comprising one or more of the polynucleotides of the present invention. Also provided is a selectable library of expression vectors comprising at least one vector encoding the subject fusion proteins.

Vectors of the present invention are generally categorized into cloning and expression vectors. Cloning vectors are useful for obtaining replicate copies of the polynucleotides they contain, or as a means of storing the polynucleotides in a depository for future recovery. Expression vectors (and host cells containing these expression vectors) can be used to obtain polypeptides produced from the polynucleotides they contain. Suitable cloning and expression vectors include any known in the art, e.g., those for use in bacterial, mammalian, yeast, insect and phage display expression systems. Suitable cloning vectors can be constructed according to standard techniques, or selected from a large number of cloning vectors available in the art. While the cloning vector selected may vary according to the host cell intended to be used, useful cloning vectors will generally have the ability to self-replicate, may possess a single target for a particular restriction endonuclease, or may carry marker genes. Suitable examples include plasmids and bacterial viruses, e.g., pBR322, pMB9, ColE1, pCR1, RP4, pUC18, mp18, mp19, phage DNAs (including filamentous and non-filamentous phage DNAs), and shuttle vectors such as pSA3 and pAT28. These and other cloning vectors are available from commercial vendors such as Clontech, BioRad, Stratagene, and Invitrogen.

Expression vectors containing these nucleic acids are useful to obtain host vector systems to produce proteins and polypeptides. In some embodiments, these expression vectors are replicable in the host organisms either as episomes or as an integral part of the chromosomal DNA. Suitable expression vectors include plasmids, viral vectors, including phagemids, adenoviruses, adeno-associated viruses, retroviruses, cosmids, etc. A number of expression vectors suitable for expression in eukaryotic cells including yeast, avian, and mammalian cells are known in the art. One example of an expression vector is pcDNA3.1 (Invitrogen, San Diego, Calif.), in which transcription is driven by the cytomegalovirus (CMV) early promoter/enhancer.

The vectors of the present invention generally comprise a transcriptional or translational control sequences required for expressing the fusion protein. Suitable transcription or translational control sequences include but are not limited to replication origin, promoter, enhancer, repressor binding regions, transcription initiation sites, ribosome binding sites, translation initiation sites, and termination sites for transcription and translation. As used herein, a “promoter” is a DNA region capable under certain conditions of binding RNA polymerase and initiating transcription of a coding region located downstream (in the 3′ direction) from the promoter. It can be constitutive or inducible. In general, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains responsible for the binding of RNA polymerase. Eukaryotic promoters will often, but not always, contain “TATA” boxes and “CAT” boxes.

The choice of promoters will largely depend on the host cells in which the vector is introduced. For animal cells, a variety of robust promoters, both viral and non-viral promoters, are known in the art. Non-limiting representative viral promoters include CMV, the early and late promoters of SV40 virus, promoters of various types of adenoviruses (e.g. adenovirus 2) and adeno-associated viruses. Suitable promoter sequences for eukaryotic cells include the promoters for 3-phosphoglycerate kinase, or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Cell-specific or tissue-specific promoters may also be used. A vast diversity of tissue specific promoters have been described and employed by artisans in the field. Exemplary promoters operative in selective animal cells include hepatocyte-specific promoters and cardiac muscle specific promoters. Depending on the choice of the recipient cell types, those skilled in the art will know of other suitable cell-specific or tissue-specific promoters applicable for the construction of the expression vectors of the present invention.

In certain preferred embodiments, the vectors of the present invention use strong enhancer and promoter expression cassettes. Examples of such expression cassettes include the human cytomegalovirus immediately early (HCMV-IE) promoter (Boshart et al, Cell 41: 521, (1985)), the β-actin promoter (Gunning et al. (1987) Proc. Natl. Acad. Sci. (USA) 84: 5831), the histone H4 promoter (Guild et al. (1988), J. Viral. 62: 3795), the mouse metallothionein promoter (McIvor et al. (1987), Mol, Cell. Biol. 7: 838), the rat growth hormone promoter (Millet et al. (1985), Mol. Cell. Biol. 5: 431), the human adenosine deaminase promoter (Hantzapoulos et al. (1989) Proc. Natl. Acad. Sci. USA 86: 3519), the HSV tk promoter 25 (Tabin et al. (1982) Mol. Cell. Biol. 2: 426), the α-1 antitrypsin enhancer (Peng et al. (1988) Proc. Natl. Acad. Sci. USA 85: 8146), and the immunoglobulin enhancer/promoter (Blankenstein et al. (1988) Nucleic Acid Res. 16: 10939), the SV40 early or late promoters, the Adenovirus 2 major late promoter, or other viral promoters derived from polyoma viris, bovine papilloma virus, or other retroviruses or adenoviruses.

In constructing the subject vectors, the termination sequences associated with the exogenous sequences are also inserted into the 3′ end of the sequence desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various polyadenylation sequences that are known in the art, widely available, and exemplified below. Where the terminator comprises a gene, it can be advantageous to use a gene which encodes a detectable or selectable marker; thereby providing a means by which the presence and/or absence of the terminator sequence (and therefore the corresponding inactivation and/or activation of the transcription unit) can be detected and/or selected.

In some embodiments, the expression vector incorporates an internal ribosomal entry site (IRES) that separates at least one domain of the fusion protein from at least one other domain of the fusion protein. Multiple IRES sequences useful in the expression of polypeptides are known to those skilled in the art, and include IRES sequences derived from hepatitis C virus, hepatitis A virus, Epstein-Barr virus, and many others. In some embodiments, an IRES separates the polynucleotide sequence encoding the B chain from the polynucleotide sequence encoding the A chain. A chains and B chains so expressed can be combined by the cell or by artificial means to form a complete human relaxin fusion protein.

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, neomycyin, G418, methotrexate, etc.; (b) complement auxotrophic deficiencies; or (c) supply critical nutrients not available from complex media. The choice of the proper marker gene will depend on the host cell, and appropriate genes for different hosts are known in the art.

### Expression Systems

In one embodiment, the invention provides a host cell comprising the recombinant polynucleodies encoding the fusion protein. In some embodiments, the polynucleotide is unincorporated into the host cell genome. In other embodiments, the polynucleotide is incorporated into the host cell genome. The expression vectors can be introduced into a suitable prokaryotic or eukaryotic host cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell. A variety of expression vector/host systems may be utilized to contain and express sequences encoding fusion proteins. These include, but are not limited to, microorganisms such as bacteria (e.g. transformed with recombinant bacteriophage, plasmid, or cosmid DNA expression vectors); yeast (e.g. transformed with yeast expression vectors); insect cell systems (e.g. infected with virus expression vectors such as baculovirus); plant cell systems (e.g. transformed with virus expression vectors such as cauliflower mosaic virus (CAMV) or tobacco mosaic virus (TMV); transformed using Agrobacterium tumefaciens-mediated transfer; or transformed with bacterial expression vectors such as Ti or pBR322 plasmids); or animal cell systems.

For most animal cells, any of the above-mentioned methods is suitable for vector delivery. Animal cells useful in the methods and compositions of the present invention include, but are not limited to, vertebrate cells, such as mammalian cells, capable of expressing exogenously introduced gene products in large quantity, e.g. at the milligram level. Non-limiting examples of preferred cells are NIH3T3 cells, COS, HeLa, and CHO cells. The animal cells can be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, animal cells can be grown in a defined medium that lacks serum but is supplemented with hormones, growth factors or any other factors necessary for the survival and/or growth of a particular cell type. Whereas a defined medium supporting cell survival maintains the viability, morphology, capacity to metabolize and potentially, capacity of the cell to differentiate, a defined medium promoting cell growth provides all chemicals necessary for cell proliferation or multiplication. The general parameters governing mammalian cell survival and growth in vitro are well established in the art. Physicochemical parameters which may be controlled in different cell culture systems include, for example, pH, pO₂, pCO₂, temperature, and osmolarity. The nutritional requirements of cells are usually provided in standard media formulations developed to provide an optimal environment. Nutrients can be divided into several categories: amino acids and their derivatives, carbohydrates, sugars, fatty acids, complex lipids, nucleic acid derivatives and vitamins. Apart from nutrients for maintaining cell metabolism, most cells also require one or more hormones from at least one of the following groups: steroids, prostaglandins, growth factors, pituitary hormones, and peptide hormones to proliferate in serum-free media (Sato, G. H., et al. in “Growth of Cells in Hormonally Defined Media”, Cold Spring Harbor Press, N.Y., 1982). In addition to hormones, cells may require transport proteins such as transferrin (plasma iron transport protein), ceruloplasmin (a copper transport protein), and high-density lipoprotein (a lipid carrier) for survival and growth in vitro. The set of optimal hormones or transport proteins will vary for each cell type. Most of these hormones or transport proteins have been added exogenously or, in a rare case, a mutant cell line has been found which does not require a particular factor. Those skilled in the art will know of other factors required for maintaining a cell culture without undue experimentation.

Plant host cells may be in the form of whole plants, isolated cells or protoplasts. Other suitable host cells for cloning and expressing the subject vectors are prokaryotes and eukaryotic microbes such as fungi or yeast cells. Suitable prokaryotes for this purpose include bacteria including Gram-negative and Gram-positive microorganisms. Representative members of this class of microorganisms are Enterobacteriaceae (e.g E. coli), Enterobacter, Erwinia, Klebsiella, Proteus, Salmonella (e.g. Salmonella typhimurium), Serratia (e.g., Sefratia marcescans), Shigella, Neisseria (e.g. Neisseria meningitidis) as well as Bacilli (e.g. Bacilli subtilis and Bacilli licheniformis). Commonly employed fungi (including yeast) host cells are S. cerevisiae, Kluyveromyces lactis (K. lactis), species of Candida including C. albicans and C. glabrata, C. maltosa, C. utilis, C. stellatoidea, C. parapsilosis, C. tropicalus, Neurospora crassas, Aspergillus nidulans, Schizosaccharomyces pombe (S. pombe), Pichia pastoris, and Yarowia lipolytica.

In one embodiment, the invention provide a method of producing a biologically active fusion protein, comprising expressing in a host cell a recombinant polynucleotide encoding a fusion protein of the invention under conditions suitable for production of said fusion protein. In some embodiments, the biological activity of the fusion protein is the same as one or more of human H1 relaxin, human H2 relaxin, or human H3 relaxin. In some embodiments, the biological activity of the fusion protein is characterized by the ability to bind to a human relaxin receptor. Non-limiting examples of human relaxin receptors include RXFP1, RXFP2, RXFP3, RXFP4, FSHR (LGR1), LHCGR (LGR2), TSHR (LGR3), LGR4, LGR5, LGR6, LGR7 (RXFP1), and LGR8 (RXFP2). In some embodiments, the conditions suitable for production of the fusion protein are substantially the same as those for the maintenance and/or growth of the host cell, as described above. In other embodiments, conditions suitable for production of the fusion protein comprise a change in the conditions for the maintenance and/or growth of the host cell. Changes to the conditions for maintenance and/or growth of the host cell include a change in one or more of a number of parameters, including but not limited to pH, temperature, concentration of one or more components of the growth or buffer media, pO₂, pCO₂, osmolarity, addition of one or more reagents (including chemical, biological, enzymatic, and other reactive agents), and addition of one or more buffers. Conditions suitable for production of the fusion protein can include conditions that support processing, cleavage, folding, assembly, and/or secretion of the fusion protein by a host cell. In addition, conditions suitable for production of the fusion protein can include conditions that support lysis of a host cell, purification of the fusion protein. Conditions suitable for production of the fusion protein can further include conditions that support processing, cleavage, folding, and/or assembly of the fusion protein outside of a host cell. In some embodiments a series of different conditions are employed to achieve two or more steps in a multi-step process culminating in the production of a biologically active fusion protein. Steps can include processing, cleavage, folding, assembly, secretion, and/or purification of the fusion protein; and/or host cell lysis. The specific conditions can be optimized for each step, and can depend on specific nature of the fusion protein, the choice of expression vector, the choice of host cell, and choice of protocol and accompanying reagents.

In one embodiment, the fusion protein expressed by a host cell is isolated. In general, isolation comprises purification of the fusion protein away from at least one other component in a mixture. Isolation can comprise separation based on characteristics such as size, charge, shape, or binding affinity of the fusion protein or a portion or domain thereof, or combined characteristics thereof. Purification can utilize a single characteristic, combinations of two or more characteristics simultaneously, or one or more characteristics in each of two or more isolation steps. Isolation by binding affinity can utilize binding affinities of the fusion protein or portion or domain thereof for a target binding partne. Alternatively, isolating the fusion protein can comprise utilizing a binding partner having specificity for the fusion protein or portion or domain thereof, such as an antibody, antibody fragment, a recombinant antibody, a non-human antibody, a chimeric antibody, a humanized antibody, or a fully human antibody. In some embodiments, a tag is included in the fusion protein that is the target of a binding agent specific for that tag, which is useful in purification of the fusion protein, wherein the tag remains as part of the fusion protein or is removed following or in the process of purification. Examples of tag/binding-partner pairs are known in the art, and include, but are not limited to His tag (e.g. 6 Histidines) and nickel, streptavidin and biotin, various epitope tags and corresponding antibodies, and Fc fragment and protein A and/or protein B. Multiple tags can be included in the fusion protein, facilitating purification using a combination of binding partners simultaneously or in sequence. An example of purifying Fc fragment-containing proteins by affinity for protein A is described, for example, by Sullam et al. (1988), Infection and Immunity 56(11): 2907-2911.

In some embodiments, the fusion protein is purified from a lysate of a host cell. The lysate can contain the fusion protein in an unprocessed form, an intermediate processed form, a fully processed form, or a mixture of fusion proteins in two or more of said forms. In other embodiments, the fusion protein is secreted from a host cell, such as into the media, and is subsequently purified. Fusion protein secreted by a host cell can be in an unprocessed form, an intermediate processed form, a fully processed form, or a mixture of fusion proteins in two or more of said forms. Processing of fusion protein by a host cell can be performed by endogenous host cell enzymes. Alternatively, a polynucleotide encoding a non-host cell enzyme for the processing of fusion protein can be introduced into a host cell, either concurrently with or in a separate process from the introduction of the polynucleotide encoding the fusion protein. An example of processing of fusion protein using enzymes introduced into a host cell is described in WO 1993/011247. In some embodiments, fusion protein is processed external to the host cell. Processing can be performed on fusion proteins in a purified or unpurified form, as well as on fusion protein that is initially unprocessed or in an intermediate processed form prior to performing additional processing. Processing can include cleavage of a polypeptide into two or more polypeptide chains and/or joining of two or more polypeptide chains. Examples of processing of fusion protein external to host cells is described in U.S. Pat. No. 5,759,807 and U.S. Pat. No. 5,464,756.

### Pharmaceutical Compositions

In one embodiment, the invention provides a pharmaceutical composition comprising the fusion protein and a pharmaceutically acceptable carrier, excipient, or stabilizer (such as described in Remington's Pharmaceutical Sciences 16th edition, Osol, A. Ed. (1980)). Generally, the pharmaceutical composition is provided as a lyophilized formulation or aqueous solution. When provided in a lyophilized formulation, the pharmaceutical composition is typically reconstituted by the addition of an aqueous component. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN J, PLURONICS J or polyethylene glycol (PEG).

The active ingredients may also be entrapped in microcapsules prepared, for example, by co-acervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate aerosolizer device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents. Buffers useful in combination with human relaxin can also be used in combination with the fusion protein. Examples of such buffers can be found in U.S. Pat. No. 5,451,572.

For parenteral administration, the fusion protein can be formulated in a unit dosage injectable form (solution, suspension, emulsion) in association with a pharmaceutically acceptable parenteral vehicle. Such vehicles are inherently nontoxic, and non-therapeutic. Examples of such vehicles are water, saline, Ringer's solution, dextrose solution, and 5% human serum albumin Nonaqueous vehicles such as fixed oils and ethyl oleate can also be used. Liposomes may be used as carriers. The vehicle may contain minor amounts of additives such as substances that enhance isotonicity and chemical stability, e.g., buffers and preservatives.

Where desired, the pharmaceutical compositions can be formulated in slow release or sustained release forms, whereby a relatively consistent level of the active compound are provided over an extended period. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

Pharmaceutical compositions can be delivered as a therapeutic or as a prophylactic (e.g., inhibiting or preventing onset of neurodegenerative diseases). Delivery as a therapeutic is aimed at providing a therapeutic benefit, by which is meant eradication or amelioration of the underlying disorder being treated. For prophylactic benefit, the agents may be administered to a patient at risk of developing a disease or to a patient reporting one or more of the physiological symptoms of such a disease, even though a diagnosis may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication. The actual amount effective for a particular application will depend, inter alia, on the condition being treated and the route of administration.

### Therapeutic Applications

As noted in the Summary, the invention provides a method for ameliorating a condition comprising administering to a subject in need thereof a composition comprising an effective amount of the fusion protein. In some embodiments, the condition is a disease or disease state; injury to an organ, tissue, or component thereof; or a combination thereof, including the conditions described in the Summary. Non-limiting examples of conditions for which the fusion protein can be administered to ameliorate include heart failure or other related or unrelated heart conditions, including acute decompensated heart failure and classes I, II, III, and IV heart failure; sinus bradycardia; neurodegenerative disease; wounds to tissues, including skin; dyspnea; ischemic wounds and other ischemic conditions; infection; hypertension; renal dysfunction; pulmonary arterial hypertension; inflammation; and fibrosis and fibromyalgia. Other conditions and applications in which the fusion protein of the present invention can find use include, but are not limited to, promoting angiogenesis, increasing the force rate of atrial contraction, increasing cardiac output, stimulating cardiac inotropy, stimulating cardiac chronotropy, restoring cardiac function following heart failure, increasing heart rate (such as to a normal level), promoting wound healing, reducing use of heart failure medications (taken concurrently or non-concurrently), increasing cardiac index, reducing hospital stay duration associated with heart failure, promoting angiogenesis, inducing secretion of vascular endothelial growth factor (VEGF), reducing hypertension, increasing vasodilation, increasing a parameter associated with a renal function, increasing the production of an angiogenic cytokine, increasing nitric oxide production in a cell (including a cell of a blood vessel), increasing endothelin type B receptor activation in a cell of a blood vessel, increasing arterial compliance, and increasing intrauterine fetal growth rate.

In some embodiments, fusion protein is administered to a subject in an amount effective to reduce the duration of hospitalization of a subject compared to a subject that does not receive fusion protein. In some embodiments, fusion protein is administered to a subject in an amount effective to reduce the projected duration of hospitalization of a subject. In some embodiments, duration of hospitalization is substantially reduced.

In one embodiment, fusion protein is administered to a subject in an amount effective to increase cardiac index. The term “cardiac index” or abbreviated “CI” describes the amount of blood that the left ventricle ejects into the systemic circulation in one minute, in relation to a subject's body size. It is a vasodynamic parameter that relates the cardiac output (CO) to body surface area (BSA) and thus relating heart performance to the size of the individual, resulting in a value with the unit of measurement of liters per minute per square meter (1/min/m²). In some embodiments, cardiac index is increased substantially. In other embodiments, cardiac index is increased measurably, as measured by the units min/m². In some embodiments, the increase in cardiac index is not accompanied by an increase in heart rate. In other embodiments, the increase in cardiac index is not accompanied by an substantial increase in heart rate greater than the subjects heart rate before treatment with fusion protein.

In one embodiment, fusion protein is administered to a subject in an amount effective to reduce at least one heart failure sign or symptom in the subject. In some embodiments, the at least one heart failure sign or symptom comprises one or more of the group consisting of dyspnea at rest, orthopnea, dyspnea on exertion, edema, rales, pulmonary congestion, jugular venous pulse or distension, edema associated weight gain, high pulmonary capillary wedge pressure, high left ventricular end-diastolic pressure, high systemic vascular resistance, low cardiac output, low left ventricular ejection fraction, need for intravenous diuretic therapy, need for additional intravenous vasodilator therapy, and incidence of worsening in-hospital heart failure. In some embodiments, reduction is by way of lowering severity, anticipated duration, or severity and anticipated duration of the at least one heart failure condition. In some embodiments, severity or anticipated duration of the at least one heart failure condition is lowered substantially.

In one embodiment, fusion protein is administered to a subject in an amount effective to reduce in-hospital worsening of heart failure in the subject. In some embodiments, the in-hospital worsening heart failure comprises one or more of worsening dyspnea, need for additional intravenous therapy to treat the heart failure, need for mechanical support of breathing, and need for mechanical support of blood pressure. In some embodiments, the method comprises reducing the 60-day risk of death or rehospitalization of the subject compared to treatment of heart failure without fusion protein. In some embodiments, the 60-day risk of death or rehospitalization is reduced substantially. In some embodiments, the method further comprises reducing the 60-day risk of rehospitalization due to heart failure or renal insufficiency of the subject compared to treatment of heart failure without fusion protein. In some embodiments, the 60-day risk of rehospitalization due to heart failure or renal insufficiency is reduced substantilly. In some embodiments, the method further comprises reducing the 180-day risk of cardiovascular death of the subject compared to treatment of heart failure without fusion protein. In another embodiment, the 180-day risk of cardiovascular death is reduced substantially.

In one embodiment, fusion protein is administered to a subject in an amount effective to treat a disease related to vasoconstriction. As used herein, the terms “disease related to vasoconstriction,” “disorder related to vasoconstriction,” “disease associated with vasoconstriction,” and “disorder associated with vasoconstriction,” used interchangeably herein, refer to a disease or condition or disorder that involves vasoconstriction in some manner. The disease may be a disease which is a direct result of vasoconstriction; a disease or condition that is exacerbated by vasoconstriction; and/or a disease or condition that is a sequelae of vasoconstriction. Diseases and disorder related to vasoconstriction include, but are not limited to: pulmonary vasoconstriction and associated diseases and disorders; cerebral vasoconstriction and associated diseases and disorders; peripheral vasoconstriction and associated diseases and disorders; cardiovascular vasoconstriction and associated diseases and disorders; renal vasoconstriction and associated diseases and disorders; and ischemic conditions. Such diseases and disorders include, but are not limited to, chronic stable angina; unstable angina; vasospastic angina; microvascular angina; blood vessel damage due to invasive manipulation, e.g., surgery; blood vessel damage due to ischemia, e.g., ischemia associated with infection, trauma, and graft rejection; ischemia associated with stroke; cerebrovascular ischemia; renal ischemia; pulmonary ischemia; limb ischemia; ischemic cardiomyopathy; myocardial ischemia; reduction in renal function as a result of treatment with a nephrotoxic agent, e.g., cyclosporine A; acute myocardial infarction; ischemic myocardium associated with hypertensive heart disease and impaired coronary vasodilator reserve; subarachnoid hemorrhage with secondary cerebral vasospasm; reversible cerebral vasoconstriction; migraine; disorders relating to uterine vascoconstriction, e.g., preeclampsia of pregnancy, eclampsia, intrauterine growth restriction, inadequate maternal vasodilation during pregnancy; post transplant cardiomyopathy; renovascular ischemia; cerebrovascular ischemia (Transient Ischemic Attack (TIA) and stroke); pulmonary hypertension; renal hypertension; essential hypertension; atheroembolic diseases; renal vein thrombosis; renal artery stenosis; renal vasoconstriction secondary to shock, trauma, or sepsis; liver ischemia, peripheral vascular disease; diabetes mellitus; thromboangiitis obliterans; and burn/thermal injury. In one embodiment, fusion protein is administered to a subject in an amount effective to reduce hypertension. In some embodiments, the hypertension is a pulmonary hypertension. In some embodiments, hypertension is reduced substantially.

In one embodiment, fusion protein is administered to a subject to increase arterial compliance. Arterial stiffness can be measured by several methods known to those of skill in the art. One measure of global arterial compliance is the AC area value, which is calculated from the diastolic decay of the aortic pressure waveform [P(t)] using the area method (Liu et al. (1986) Am. J. P̂rø/0.251:H588-H600). Another measure of global arterial compliance is calculated as the stroke volume to pulse pressure ratio (Chemla et al. (1998) Am. J. Physiol 274:H500-H505). Local arterial compliance can be determined by measuring the elasticity of an arterial wall at particular point using invasive or non-invasive means. See, e.g., U.S. Pat. No. 6,267,728. Regional compliance, which describes compliance in an arterial segment, can be calculated from arterial volume and distensibility, and can be measured with the use of pulse wave velocity. See, e.g., Ogawa et al, Cardiovascular Diabetology (2003) 2:10; Safar et al, Arch Mal Coer (2002) 95:1215-18. Other suitable methods of measuring arterial compliance are described in the literature, and any known method can be used. See, e.g., Cohn, J. N., “Evaluation of Arterial Compliance”, In: Hypertension Primer, Izzo, J. L. and Black, H. R., (eds.), Pub. by Council on High Blood Pressure Research, American Heart Association, pp. 252-253, (1993); Finkelstein, S. M., et al., “First and Third-Order Models for Determining Arterial Compliance”, Journal of Hypertension, 10 (Suppl. 6) S11-S14, (1992); Haidet, G. C., et al., “Effects of Aging on Arterial Compliance in the Beagle”, Clinical Research, 40, 266A, (1992); McVeigh, G. E., et al., “Assessment of Arterial Compliance in Hypertension”, Current Opinion in Nephrology and Hypertension, 2, 82-86, (1993). In some embodiments, arterial compliance is increased substantially.

In one embodiment, fusion protein is administered to a subject in an amount effective to treat fibrosis. The term “fibrosis” includes any condition characterized by the formation or development of excess fibrous connective tissue, excess extracellular matrix, excess scarring or excess collagen deposition in an organ or tissue as a reparative or reactive process. Fibrosis related diseases include, but are not limited to: idiopathic pulmonary fibrosis; skin fibrosis, such as scleroderma, post-traumatic and operative cutaneous scarring; eye fibrosis, such as sclerosis of the eyes, conjunctival and corneal scarring, pterygium; cystic fibrosis of the pancreas and lungs; endomyocardial fibrosis; idiopathic myocardiopathy; cirrhosis; mediastinal fibrosis; progressive massive fibrosis; proliferative fibrosis; neoplastic fibrosis. Tuberculosis can cause fibrosis of the lungs. Therefore, the present invention can be used to treat fibrosis in a wide range of organs and tissues, including, but not limited to, the lung, eye, skin, kidney, liver, pancreas and joints. In some embodiments, fusion protein is administered to a subject in an amount effective to alleviate, reduce in severity and/or duration, or otherwise ameliorate a sign, symptom, or consequence of fibrosis. Signs, symptoms, and consequences of fibrosis vary with the tissue affected. Signs or clinical symptoms of lung fibrosis include, but are not limited to increased deposition of collagen, particularly in alveolar septa and peribronchial parenchyma, thickened alveolar septa, decreased gas exchange resulting in elevated circulating carbon dioxide and reduced circulating oxygen levels, decreased lung elasticity which can manifest as restrictive lung functional impairment with decreased lung volumes and compliance on pulmonary function tests, bilateral reticulonodular images on chest X-ray, progressive dyspnea (difficulty breathing), and hypoxemia at rest that worsens with exercise. Signs and symptoms associated with liver fibrosis include, but are not limited to, jaundice, skin changes, fluid retention, nail changes, easy bruising, nose bleeds, male subjects having enlarged breasts, exhaustion, fatigue, loss of appetite, nausea, weakness and/or weight loss. In some embodiments, fibrosis is reduced substantially. In some embodiments, one or more fibrosis assessment criteria is improved substantially. In still other embodiments, one or more signs, symptoms, or conditions of fibrosis is reduced in severity or duration by a substantial amount.

In one embodiment, the methods of the invention provide hemodynamic effects consistent with vasoldialtion, including improved parameters reflecting renal function in subjects with stable compensated chronic heart failure (HF). The dosage schedule and amounts effective for this and other uses in a variety of conditions, i.e., the “dosing regimen,” will depend upon a variety of factors, including the stage of the disease or condition, the severity of the disease or condition, the severity of the adverse side effects, the general state of the patient's health, the patient's physical status, age and the like. In calculating the dosage regimen for a patient, the mode of administration is also taken into consideration. The dosage regimen must also take into consideration the pharmacokinetics, i.e., the rate of absorption, bioavailability, metabolism, clearance, and the like. Based on those principles, the fusion protein can be used to treat human subjects diagnosed with symptoms of heart failure to maintain stable compensated chronic HF.

In one embodiment, the invention provides a fusion protein and additional drugs, including but not limited to antiplatelet therapy, beta-blockers, diuretics, nitrates, hydralazine, inotropes, digitalis, and angiotensin-converting enzyme inhibitors or angiotensin receptor blockers for simultaneous, combined, separate or sequential administration. The invention also provides the use of antiplatelet therapy, beta-blockers, diuretics, nitrates, hydralazine, inotropes, digitalis, and angiotensin-converting enzyme inhibitors or angiotensin receptor blockers in the manufacture of a medicament for managing stable compensated chronic HF, wherein the medicament is prepared for administration with the fusion protein.

Further contemplated is the use of the fusion protein in the manufacture of a medicament for managing stable compensated chronic HF, wherein the patient has previously (e.g., a few hours before, one or more days, weeks, or months, or years before, etc.) been treated with antiplatelet therapy, beta-blockers, diuretics, nitrates, hydralazine, inotropes, digitalis, and angiotensin-converting enzyme inhibitors or angiotensin receptor blockers. In one embodiment, one or more of the drugs such as, antiplatelet therapy, beta-blockers, diuretics, nitrates, hydralazine, inotropes, digitalis, and angiotensin-converting enzyme inhibitors or angiotensin receptor blockers are still active in vivo in the patient. The invention also provides the use of antiplatelet therapy, beta-blockers, diuretics, nitrates, hydralazine, inotropes, digitalis, and angiotensin-converting enzyme inhibitors or angiotensin receptor blockers in the manufacture of a medicament for managing stable compensated chronic HF, wherein the patient has previously been treated with the fusion protein.

The state of the art allows the clinician to determine the dosage regimen of the fusion protein for each individual patient. As an illustrative example, the guidelines provided below for fusion protein dosing can be used as guidance to determine the dosage regimen, i.e., dose schedule and dosage levels, of formulations containing pharmaceutically active fusion protein administered when practicing the methods of the invention. In one embodiment, the daily dose of pharmaceutically active fusion protein is in an amount in a range of about 10 to 960 mcg/kg of subject body weight per day. In one embodiment, the dose of fusion protein is 10, 30, or 100 mcg/kg/day. In another embodiment, the dosage of fusion protein is 240, 480, or 960 mcg/kg/day. In another embodiment, the dose of fusion protein needed to achieve a desired effect is substantially lower than the dose of human relaxin lacking the constant immunoglobulin domain required to achieve the same effect. In another embodiment, administration of fusion protein is continued so as to maintain a serum concentration of fusion protein from about 0.01 to about 500 ng/ml, for example from about 0.01 ng/ml to about 0.05 ng/ml, from about 0.05 ng/ml to about 0.1 ng/ml, from about 0.1 ng/ml to about 0.25 ng/ml, from about 0.25 ng/ml to about 0.5 ng/ml, from about 0.5 ng/ml to about 1.0 ng/ml, from about 1.0 ng/ml to about 5 ng/ml, from about 5 ng/ml to about 10 ng/ml, from about 10 ng/ml to about 15 ng/ml, from about 15 ng/ml to about 20 ng/ml, from about 20 ng/ml to about 25 ng/ml, from about 25 ng/ml to about 30 ng/ml, from about 30 ng/ml to about 35 ng/ml, from about 35 ng/ml to about 40 ng/ml, from about 40 ng/ml to about 45 ng/ml, from about 45 ng/ml to about 50 ng/ml, from about 50 ng/ml to about 60 ng/ml, from about 60 ng/ml to about 70 ng/ml, or from about 70 ng/ml to about 80 ng/ml, or from about 3 to about 300 ng/ml. Thus, the methods of the present invention include administrations that result in these serum concentrations of the fusion protein. In some embodiments, these fusion protein concentrations are used to ameliorate or reduce decompensation events such as dyspnea, hypertension, high blood pressure, arrhythmia, reduced renal blood flow, renal insufficiency and mortality. In a further embodiment, these fusion protein concentrations are used to ameliorate or reduce neurohormonal imbalance, fluid overload, cardiac arrhythmia, cardiac ischemia, risk of mortality, cardiac stress, vascular resistance, and the like. Depending on the subject, the fusion protein administration is maintained for a specific period of time or for as long as needed to maintain stability in the subject.

The duration of treatment with a fusion protein of the present invention can be indefinite for some subjects. In some embodiments, where the pharmaceutical composition comprising the fusion protein is administered intravenously, duration can be limited to a range, such as between 1 hour and 96 hours depending on the patient, and one or more optional repeat treatments as needed. For example, with respect to frequency of administration, fusion protein administration can be a continuous infusion lasting from about 1 hour to 48 hours of treatment. The fusion protein can be given continuously or intermittent via intravenous or subcutaneous administration (or intradermal, sublingual, inhalation, or by wearable infusion pump). For intravenous administration, fusion protein can be delivered by syringe pump or through an IV bag. The IV bag can be a standard saline, half normal saline, 5% dextrose in water, lactated Ringer's or similar solution in a 100, 250, 500 or 1000 ml IV bag. For subcutaneous infusion, fusion protein can be administered by a subcutaneous infusion set connected to a wearable infusion pump. Depending on the subject, the fusion protein administration is maintained for as specific period of time (e.g. 4, 8, 12, 24, and 48 hours) or, administered intermittently, for as long as needed (e.g. daily, monthly, or for 7, 14, 21 days etc.) to maintain stability in the subject.

Some subjects are treated indefinitely while others are treated for specific periods of time. It is also possible to treat a subject on and off with fusion protein as needed. Thus, administration can be continued over a period of time sufficient to maintain a stable compensated chronic HF resulting in an amelioration or reduction of fibrosis or acute cardiac decompensation events, including but not limited to, dyspnea, hypertension, high blood pressure, arrhythmia, reduced renal blood flow and renal insufficiency. The formulations should provide a sufficient quantity of fusion protein to effectively ameliorate and stabilize the condition. A typical pharmaceutical formulation for intravenous administration of fusion protein would depend on the specific therapy. For example, fusion protein may be administered to a patient through monotherapy (i.e., with no other concomitant medications) or in combination therapy with another medication such as antiplatelet therapy, beta-blockers, diuretics, nitrates, hydralazine, inotropes, digitalis, and angiotensin-converting enzyme inhibitors or angiotensin receptor blockers or other heart-related drug, or other fibrosis-related drug (including anti-inflammatory drugs). In one embodiment, fusion protein is administered to a patient daily as monotherapy. In another embodiment, fusion protein is administered to a patient daily as combination therapy with another drug. Notably, the dosages and frequencies of fusion protein administered to a patient may vary depending on age, degree of illness, drug tolerance, and concomitant medications and conditions. In a further embodiment fusion protein is administered to a patient with the ultimate goal to replace, reduce, or omit the other medications to reduce their side effects and to increase or maintain the therapeutic benefit of medical intervention using the fusion protein in order to optimally maintain a stable, compensated, and chronic heart failure.

In addition, the treatment duration and regimen can vary depending on the particular condition and subject that is to be treated. For instance, a therapeutic agent can be administered by the subject method over at least 1, 7, 14, 30, 60, 90 days, or a period of months, years, or even throughout the lifetime of a subject. Doses of the pharmaceutical composition comprising the fusion protein can be administered one or more times a day; once every 1, 2, 3, 4, 5, 6, 7, 8, 9, 10, 11, 12, 13, 14, or more days; once every 1, 2, 3, 4, 5, 6, 7, 8, or more weeks; or in periodic combinations as needed, such as multiple times a day for a number of weeks, followed by a period of time without such treatment.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Production of a Human Relaxin Fusion Protein

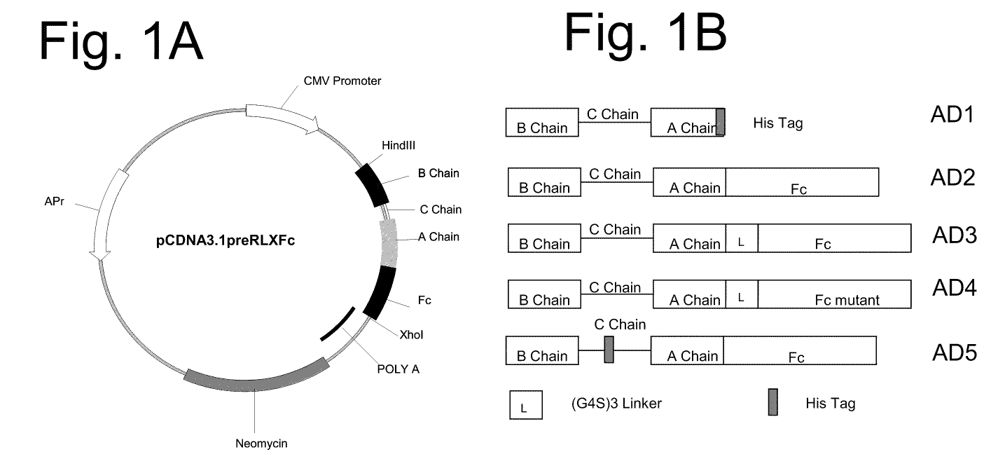

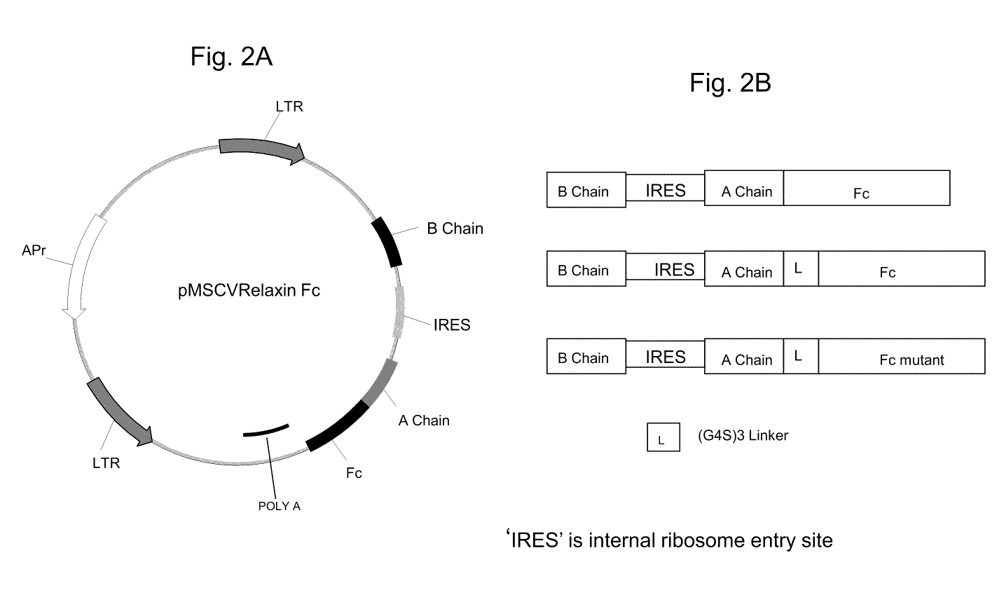

Expression vectors encoding fusion proteins of the present invention can be produced by a number of techniques known in the art, including chemical synthesis, recombinant cloning methods, PCR, combinations thereof. A polynucleotide encoding human relaxin can first be obtained as a cDNA, and subsequently manipulated for inclusion in an expression vector as part of a fusion protein. A constant immunoglobulin domain, such as an Fc fragment, can be similarly obtained and manipulated. Example expression vectors produced by such manipulations are illustrated in FIG. 1A, 1B and FIG. 2A, 2B. In FIGS. 1A and 1B, polynucleotides encoding a fusion protein having the A, B, and C chains of a human relaxin, an Fc fragment, and optionally a linker are introduced into the commercially available plasmid pcDNA3.1 (Invitrogen, San Diego, Calif.). One polynucleotide encoding a fusion protein is incorporated into the illustrated plasmid, with the same and alternative polynucleotide encoding fusion proteins illustrated beneath it. In addition to elements introduced into the plasmid, pcDNA3.1 also contains a CMV promoter, a polyadenylation signal (poly A), and genes capable of providing a host cell with resistance to ampicillin (AP^{r}) and neomycin. In FIGS. 2A and 2B, polynucleotides similar to those in FIGS. 1A, 1B are introduced into the commercially available plasmid pMSCV (Clontech, Mountain View, Calif.). One polynucleotide encoding a fusion protein is incorporated into the illustrated plasmid, with the same and alternative polynucleotide encoding fusion proteins illustrated beneath it. The difference between the fusion protein-expressing polynucleotides in FIGS. 2A and 2B and those in FIGS. 1A, 1B is that in FIGS. 2A and 2B, an internal ribosomal entry site (IRES) replaces the C chain of human relaxin. In addition to elements introduced into the plasmid, pMSCV also contains long terminal repeats (LTRs) for driving expression and permitting optional packaging into retroviral particles, as well as a gene capable of providing a host cell with resistance to ampicillin (AP^{r}).

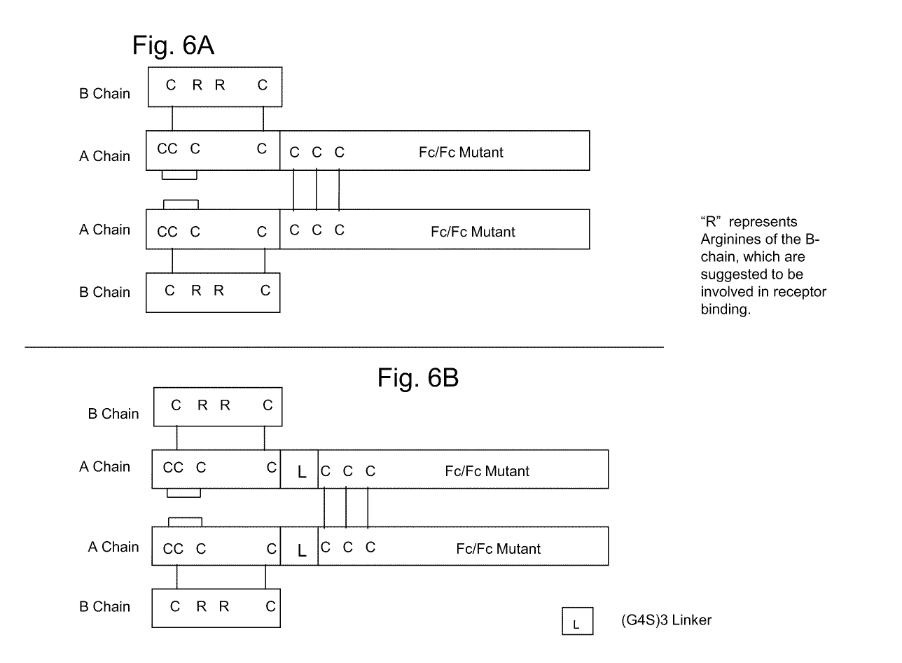

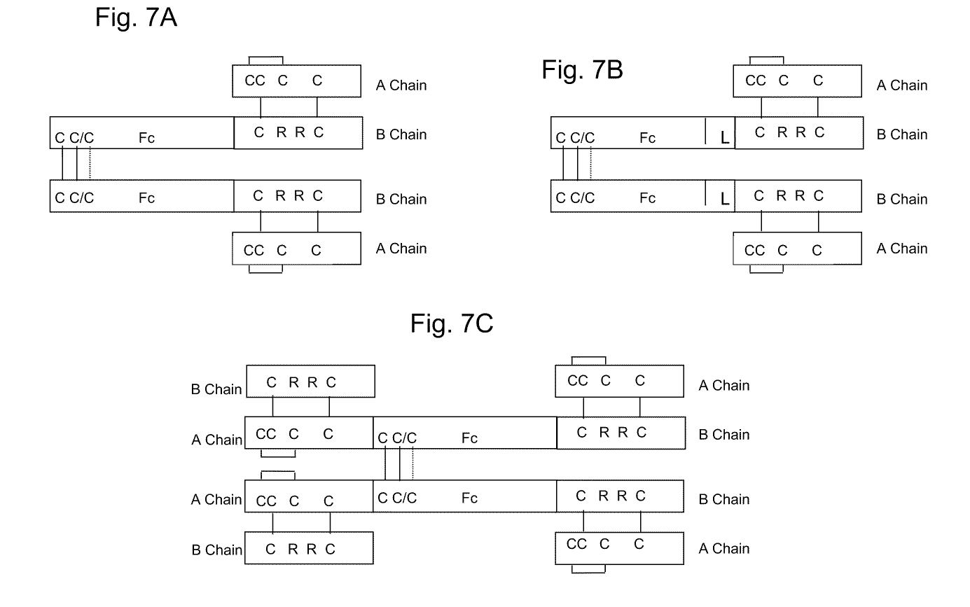

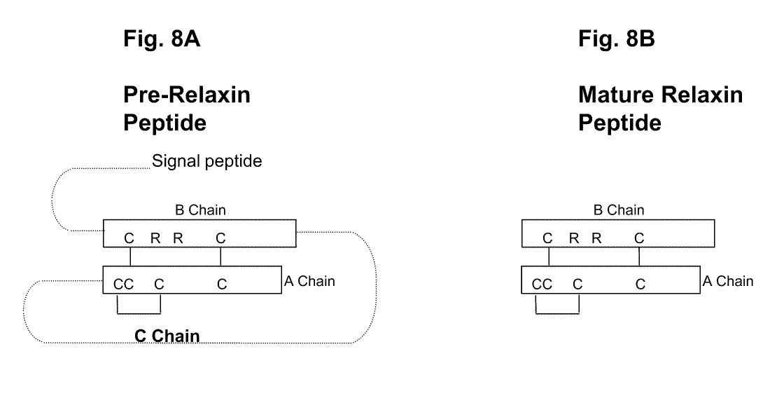

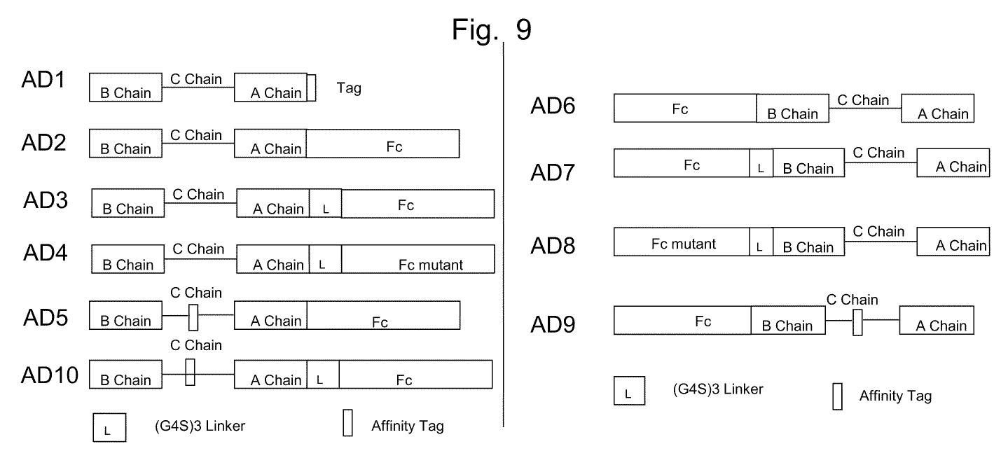

The amino acid sequences of exemplary domains of fusion proteins contemplated by the present invention are provided in FIGS. 3A,3B and 3C. FIG. 3A provides the amino acid sequence of human H2 relaxin, including, from the N-terminus to the C-terminus, signal peptide, B chain, C chain, A chain. FIGS. 3B and 3C provide, respectively, the amino acid sequence, from N-terminus to C-terminus, of an exemplary Fc-γ1 fragment and an Fc-γ4 fragment. Possible combinations of these two elements in the formation of fusion proteins are illustrated in FIGS. 4-6, with each accompanied by its amino acid sequence. In FIG. 4A, the fusion protein lacks a constant immunoglobulin domain, replacing it instead with the addition of six histidine residues, and can serve as an easily purified human relaxin control tag for fusion proteins having the constant immunoglobulin domain. In FIG. 4B, a Fc-γ1 fragment is fused directly to the A chain of human H2 relaxin, and FIG. 4C shows a Fc-γ4 fragment so fused. In FIG. 5A, a linker sequence (itallics) is introduced between the human H2 relaxin A chain and the Fc-γ1 fragment of the fusion protein. FIG. 5C is like FIG. 5A but with the Fc-γ4 fragment. In FIG. 5B, in addition to a linker sequence (itallics) as in FIG. 7, two mutations (bold) that result in changing a threonine to a glutamine (T to Q) and a methionine to a leucine (M to L) are introduced, changes which are associated with increased serum half-life in antibodies (Hinton P R. et al. (2004), J Biol Chem. 279(8):6213-6).

Chinese hamster ovary (CHO) cells can be transfected with the expression plasmid illustrated in FIG. 1. Transfection can be by any number of methods known in the art, as described above. For example, CHO cells, grown and maintained using standard methods, can be transfected by electroporation or liposomes. Following electroporation or liposomes, cells can be allowed to recover in non-selective media for 1 day, after which selection is applied by adding G418 to the growth medium. Cells containing at least one copy of the plasmid are allowed to proliferate. For transient tranfection system, adding selection agent G418 is optional for target protein production. After 2-5 days post-transfection, media is collected and fusion protein that was expressed, processed, and secreted is isloated, for example by binding to and eluting from a binding affinity column of protein A. Similar procedures can be followed to generate a stably transfected CHO cell line, which allows longer incubation time, easier scale-up, and higher production levels of fusion protein. Methods for generating stably transfected cell lines, including CHO cell lines, are well known in the art. The CHO transfection can follow the same protocol as in the transient expression system for 293 cells, described below.

### Example 1A

### Production in a Transient Transfection System

### Material and Methods:

Freestyle 293 Expression System (Invitrogen, Cat #: K9000-01) is used for transient transfection. The following protocol is followed when transfection of 100 ml of Freestyle 293 suspension cells using 293fectin,
Step 1: Pre-warm Freestyle 293 Max media and Opti-MEM media to 37° C.
Step 2: Add 200 μl 293fectin (Invitrogen, Cat. No. 12347-019) into 3.3 ml Opti-MEM and mix gently. Let incubate at room temperature for 5 min
Step 3: Add 100 μg DNA into Opti-MEM to a total volume of 3.5 ml (ex. 175 ul DNA into 3.325 ml Opti-MEM). Mix gently.
Step 4: After 5 min 293fectin incubation, add diluted DNA (from step 3) into diluted 293fectin (from Step 2). Mix gently and let incubate 30 min at room temperature.
Step 5: Dilute cells with Freestyle 293 media to 1×10⁶ cells/ml.
Step 6: After 30 min DNA+293fectin incubation, add DNA complex (step 4) into spin flask of cells (step 5). Shake at 100 rpm in incubator at 37° C. with 7.5% CO₂ for 2-3 days.

### Analysis of Protein Expression

3 ml cell suspension is collected after 3 day transfection. The supernatant is harvested after the cells are spin down, and concentrated by 10× in centrifugal filter (50 KD MWCO) at 3500 rpm for 5 min 5 μl each sample is load in 4-12% bis-tris SDS-PAGE gels for Coomassie Stain and Western Blot analyses; where Western Blot protocol was as follows:

### Electrophoretic Separation (SDS-PAGE)

a. Pour 1×SDS-PAGE Running Buffer into the Western Blot tank.
b. Position the gels in the gel holder assembly and immerse into the tank.
c. Fill the inner compartment (between the two gels) with SDS-PAGE Buffer.
d. Carefully load the samples in the wells (using a fine-tipped pipette).
e. Place the lid on the tank and plug it into the power source.
f. Run the apparatus at 125V until the samples have passed the stacking gel.
g. Turn the voltage up to 160V and allow the samples time to separate; use a pre-stained molecular weight marker to determine the end-point of the electrophoresis.

### Transfer Protocol

1. Cut filter paper in approximately 7×20 cm pieces; cut PVDF membrane to 7×20 cm.
2. Pre wet the PVDF membrane using 100% methanol for 10 seconds and immerse in dH2O, Soak the filter pads in PVDF Transfer Buffer.
3. Assemble the membrane sandwich according to the kit instructions;
4. Fill the transfer tank with 1× transfer buffer.
5. Run the transfer protocol at 25 mA (constant amperage) for 1-2 hour.

### Western Blot

Block the membrane in 5% Non Fat Dry Milk (NFDM) in PBST for 1-2 hours.

Incubate the membrane with the primary antibody for 2-16 hours at 4° C.

For Human IgG1 Fc detection, used Sigma B3773 (Monoclonal Anti-Human IgG-Fc specific-Biotin, 1:2000 dilution) and, A0170 (goat aHuFc-Perosidase specific to human, 1:50 k dilution). Both antibodies worked well.

Dilute the antibody with a 2.5% Non Fat Dry Milk (NFDM) in Tween TBS solution. A total antibody and diluent solution of 5 ml will coat a small membrane in a rotating tube well.

Remove the antibody and perform 5× washes (10 minutes of rotation each) with Tween TBS. Re-block the membrane in 10% Non Fat Dry Milk (NFDM) in Tween TBS for 10 minutes at room temperature.

Incubate the membrane with the secondary antibody for 30 minutes at room temperature. Dilute the antibody with a 2.5% Non Fat Dry Milk (NFDM) in Tween TBS solution. One can also add blocking serum from the same species in which the secondary antibody was produced.

Remove the antibody and wash 5× with Tween TBS.

Prepare the chemiluminscent reagents. It is important to prepare the ECL solution just prior to use in order to maximize its effectiveness.

Pour the chemiluminscent solution over the membrane, covering it completely.

Turn out the lights and place the membrane/acetate sandwich in a film cassette with the appropriate film. Exposure times are extremely variable and some care should be taken to determine the optimal exposure parameters. Develop film; remember to use a fixing solution.

### Example 2

### Process of Making of Human Relaxin-Linker-Fc and Human Relaxin-Fc Fusion Protein

Step 1: Transfect cells (293 cells) with expression vector plasmid DNA (see FIGS. 1 and 2).
Step 2: Grow cells in serum free media.
Step 3: Collect supernatant on day 3.
Step 4: Purify the human relaxin-linker-Fc fusion proteins using a Protein-G column (see Example 3 below)—the desired protein (containing an Fc fragment) will bind to Protein G column at binding conditions (pH 7-7.4), and will be eluted at low pH conditions (pH 2.7).
Step 5: Clean C-chain by running the eluted product through an affinity tag binding column. The eluted proteins from step 4 contain a mixture of human relaxin-linker-Fc with the C-chain of relaxin uncleaved and human relaxin-linker-Fc with cleaved C-chain. The C-chain has an affinity tag (Histidine) (see AD5, AD9 and AD10 of FIG. 9), so the human relaxin-linker-Fc with the un-cleaved C-chain will bind to the affinity column. The portion passing through includes the desired protein.
Step 6: The product from step 5 is characterized by SDS-page and gel analysis.

### Results:

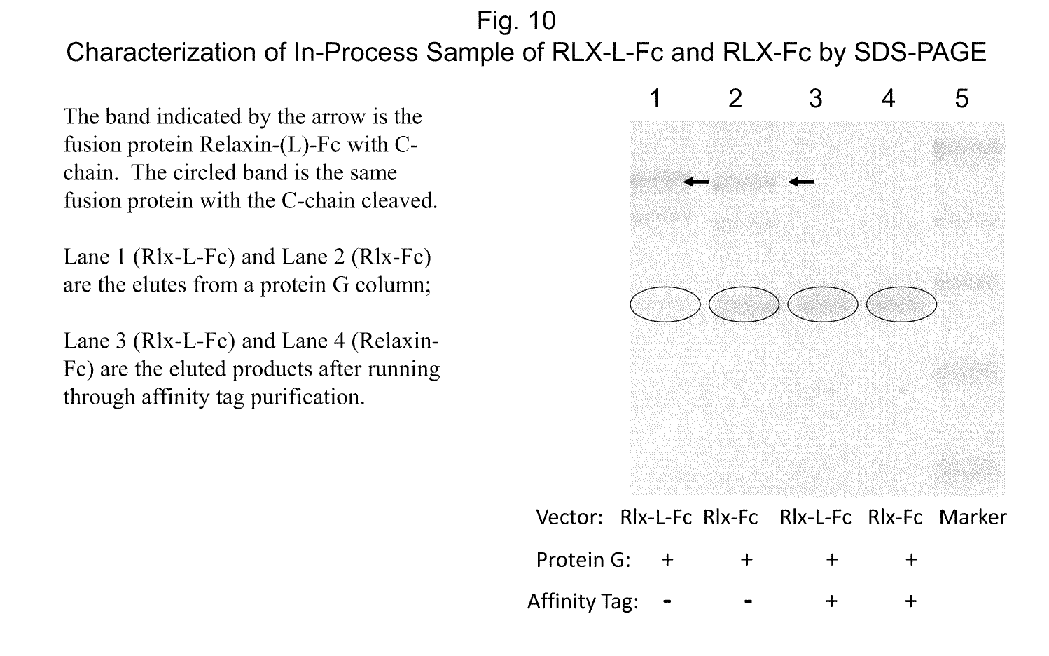

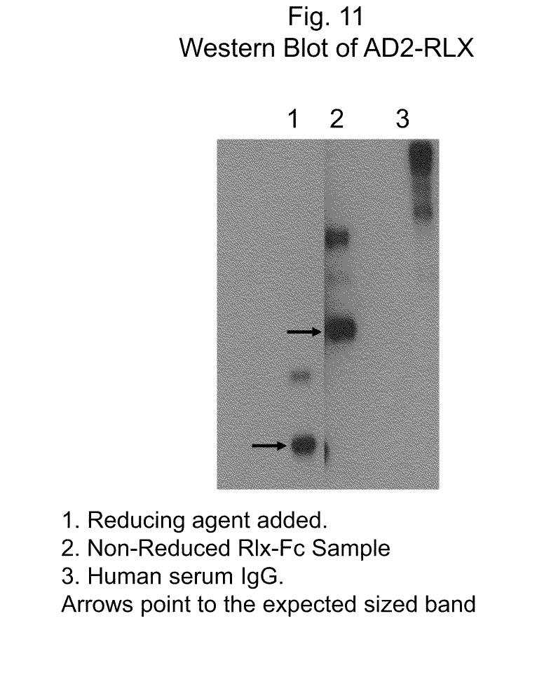

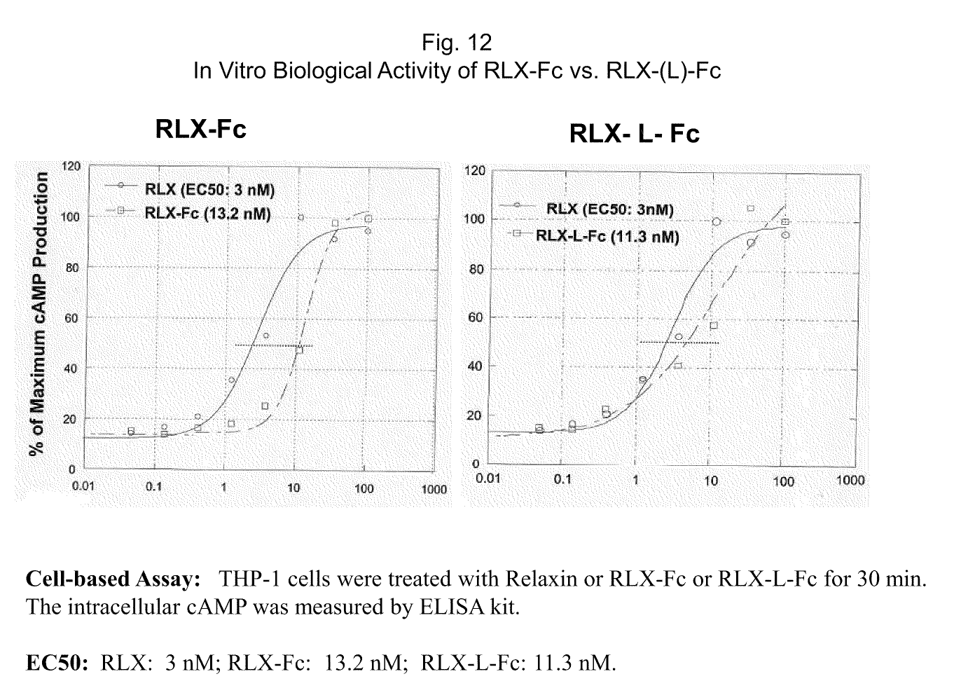

FIG. 10 is an example of SDS-PAGE with samples from expressed human relaxin-Fc. Distinct bands represent human relaxin-linker-Fc with the C-chain of relaxin uncleaved and human Relaxin-(L)-Fc with cleaved C-chain can be seen on SDS-page. Lane 1 and Lane 2 are the elutes from protein G column. Lane 3 and 4 on the SDS-page represent affinity tag purified human relaxin-(L)-Fc with cleaved C-chain and human relaxin-Fc with cleaved C-chain show more product present.

### Example 3

### Purification of Fc Fragment Using Protein G Column

Human Fc fragment can be purified using a Protein G Column or in a Quantitative Assay as described below in Example 3A.

### Column: GE Hi-Trap Protein G HP (17-0404-01)

### FPLC: Pharmacia

### Binding Buffer: PBS pH 7.4 or 20 mM Sodium Phosphate pH 7.0

### Elution Buffer: 0.1 M Glycine-HCl, pH 2.6

Neutralization buffer: 1 M Tris-HCl, pH 9.0
1. Sample preparation:

Collect cell culture media after 2-3 days of transfection, spin at 3000 rpm for 20 min, and collect supernatant.

2. Adjust the supernatant with 10×PBS. Example: for 100 mL media from step 1, add 11 mL 10×PBS.
3. Run FPLC Protein G column (This FPLC protocol can be modified to run manually using syringe) Pumping Binding Buffer into column at a rate 1 mL/min for 30 min
4. Pump Sample solution from Step 2 into column at a rate 1-1.5 mL/min
5. Wash column with Binding Buffer at 1 mL/min for 15-20 min
6. Elute the column with Elution buffer at 1 mL/min; and collect 1 mL of elutions into tubes pre-filled with 100 uL Neutralization buffer (1 M Tris-HCl, pH 9.0). Collect total about 20 mL of elution samples. Column will be run through elution buffer for 30 min, and water 30 min, and 20% ethanol 30 min

### Example 3A

### Human IgG-Fc Quantitative ELISA Protocol

### Assay Conditions:

The assay has been tested for the protocol and materials listed below using standard dilutions of human IgG Fc in the 2-400 ng/ml range. The operator must determine appropriate dilutions of reagents for alternative assay conditions.

### Example 3A

### Human IgG Fc Fragment Quantitative ELISA Protocol

### Buffer Preparation

Prepare the following buffers:

### A. Coating Buffer, 0.05 M Carbonate-Bicarbonate, pH 9.6

### B. Wash Solution: 0.05% Tween 20 in PBS, pH 7.4

### C. Blocking Solution, 50 mM Tris, 0.14 M NaCl, 1% BSA, pH 8.0

### D. Sample/Conjugate Diluents, 50 mM Tris, 0.14 M NaCl, 1% BSA, 0.05% Tween 20, pH 8.0

### E. Enzyme Substrate, TMB (KPL, Cat #50-76-00)

F. Stopping Solution, 2 M H2504 or other appropriate solution

### Step-By-Step Method (Perform all Steps at Room Temperature)

1. Coating with Capture Antibody
A. Dilute Capture Antibody (Sigma 12136) in 11 ml coating buffer to make a 1:500 dilution.
B. Add 50 ul ul per well.
C. Incubate coated plate for 60 minutes.
D. After incubation, aspirate the Capture Antibody solution from each well.
E. Wash each well with Wash Solution as follows:

Fill each well with Wash Solution; Remove Wash Solution; Repeat 8 washes.

### 2. Blocking (Post-coat)

A. Add 200 ul of Blocking Solution to each well; Incubate for 60 minutes.
B. After incubation, remove the Blocking Solution and wash each well 6×.

### 3. Standards and Samples

A. Prepare serial dilutions at a 1:2 ratio (400-6.25 ng/ml). Add 50 ul to each well.
B. Dilute the samples (in PBS), based on the expected concentration, to fit within the range of the standards. Add 50 ul diluted sample to well.
C. Incubate plate for 60 minutes. After incubation, wash each well 8 times.

### 4. Detection Antibody—Horseradish Peroxidase Conjugate

A. Dilute HRP conjugate (Sigma A0170) in 12 ml Conjugate diluent to make a 1:20000 dilution.
B. Transfer 50 ul to each well; Incubate for 60 minutes.
C. After incubation, remove HRP Conjugate and wash each well 8 times.

### 5. Enzyme Substrate Reaction

A. Prepare the Substrate solution.
B. Transfer 50 ul of Substrate solution to each well.
C. Incubate plate for 5-30 minutes.
D. To stop reaction, add 50 ul of 2 M H2SO4 to each well.

### 6. Plate Reading

Using a microtiter plate reader, read the plate at the wavelength that is appropriate (450 nm for TMB).

### Coating Buffer

Dissolve 5.3 g of Na₂CO₃ in 900 ml distilled H₂O.
Dissolve 4.2 g of NaHCO₃ in the solution from step 1.
Dissolve 1 g sodium azide in the solution from step 2 (optional) pH to 9.6
Adjust volume to 1 L with additional distilled H₂O.

### Example 4

Cell-based Assay to measure relaxin biological activity: THP-1 cells were treated with human relaxin or human relaxin-Fc or human relaxin-L-Fc for 30 min. The intracellular Adenosine 3′,5′-cyclic monophosphate (cAMP) was measured by ELISA kit. Based on the assay results, as shown in FIG. 12, the EC50 for the three different products were determined to be: RLX: 3 nM; RLX-Fc: 13.2 nM; RLX-L-Fc: 11.3 nM. The protocol is as follows:

Serum starve THP1 cells and plate cells on 1×10⁶ per well in a 12-well plate.
Treat cells with various concentration of RLX or RLX-Fc, or RLX-L-Fc or mock in the absence or presence of IBMX 250 uM for 30 min
IBMX (3-Isobutyl-1-methylxanthine, Sigma, I7018) is a non-specific inhibitor of cAMP and cGMP phosphodiesterases.
Make working solutions prior to experiments: RLX stock: 1.5 mg/ml. Dilute stock in cell culture media 217×: (5 μl stock to 1.087 mL media), the final is RLX 1 μlM (1 μM=6.9 μg/ml). For RLX-Fc, 1 μM=69 μg/mL.
RLX Dilution: For EC50 determination, starting concentration is 1000 nM (6900 ng/ml), make a series of 3× dilution from 1000 nM to 0.45 nM (total 8 dilutions) in 96-well plate. Add 1.1/10 of the volume to cell culture media (example: add 110 μl of diluted RLX to 1 mL cell culture media).

Prepare Cell Lysates from cell culture as following:

1. Wash cells three times in cold PBS.
2. Resuspend cells in Cell Lysis Buffer 5 (1×) to a concentration of 1×10⁷ cells/mL.
3. Freeze cells at −20° C. Thaw cells with gentle mixing. Trypan Blue and a microscope can be used to confirm cell lysis. Repeat freeze/thaw cycle as needed.
4. Centrifuge at 600×g for 10 minutes at 2-8° C. to remove cellular debris.
5. Assay the supernate immediately or aliquot and store at −20° C.

Measure cAMP level using an ELISA kit (Sigma # CA2000 or R&D # SKGE002B) according to manufacturer's instruction.

### Example 5

### Determining the Pk of Human Relaxin, Human Relaxin-Fc and Human Relaxin-L-Fc

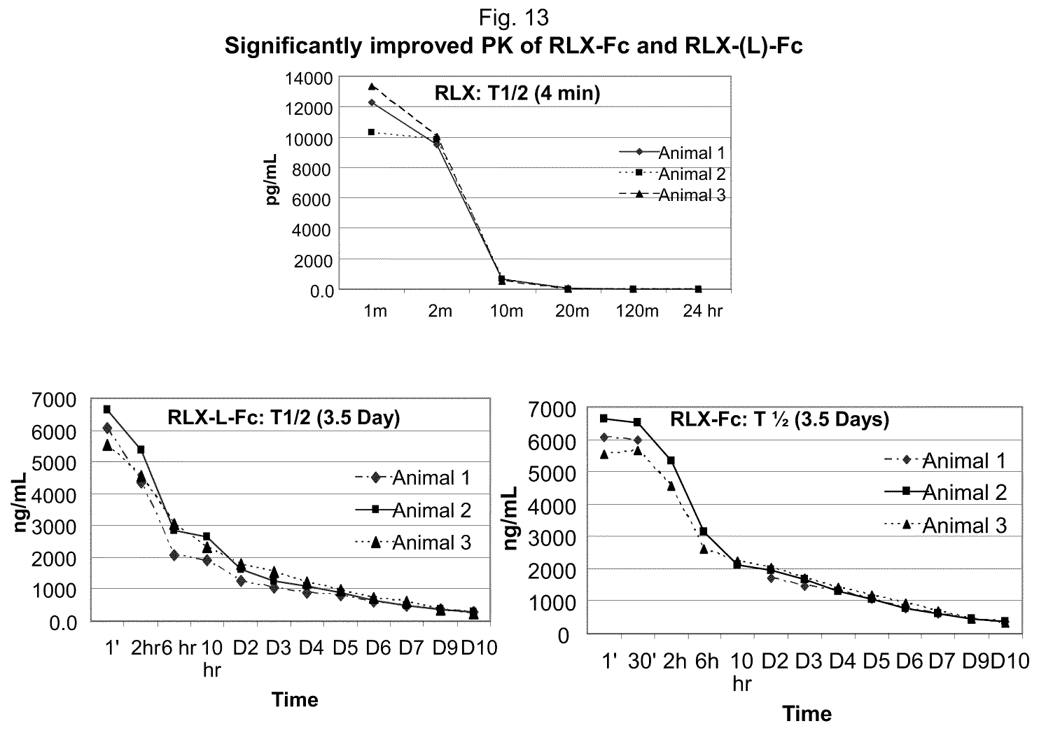

Jugular vein catheterized rats were obtained from Charles River laboratory (Wilmington, Mass.). The animals were surgically implanted with a catheter that allows repeated blood sampling. Relaxin, Relaxin-Fc or Relaxin-(L)-Fc was administered to the animals through tail vein injection. At different time points, as indicated in FIG. 13, 300-500 μl blood was withdrawn. Serum was collected and frozen at −80° C. for future relaxin, relaxin-Fc or relaxin-(L)-Fc measurements.

The level of relaxin in animal serum was measured using an ELISA kit (Immundiagnostik AG, Germany). The level of relaxin-(L)-Fc was determined using a relaxin ELISA kit and confirmed by a human Fc ELISA assay. The human Fc ELISA assay was conducted by using a coated capture antibody (Sigma I2136) and detection antibody (Sigma A0170), and following the standard ELISA protocol. As seen in FIG. 13, relaxin-Fc and relaxin-(L)-Fc had a significantly longer Pk than relaxin.

### Example 6

### Anti-Fibrotic Effects of Long-Lasting Relaxin Fusion Protein in Belomycin-Induced Lung Fibrosis Model in Murine

Fibrosis involves excessive deposition of extracellular matrix, especially collagen, by the cells that constitute the functional elements of tissues and organs. Fibrosis leads to derangement in the three-dimensional structure of organs such that the specialized cells of the organ lose functional capacity and eventually fail. Fibrosis is not only the result of necrosis or tissue breakdown, a type of scarring, but also the result of derangement in the coordinated synthesis and degradation of matrix by cells that are responsible for maintaining the unique scaffolding of such organs. Fibrosis is the end result of a variety of insults (inflammation, infections, metabolic disease, or unknown insults) and occurs commonly in organs such as lung, liver, heart, skin, and kidney. Currently, there are no FDA approved therapies that directly target fibrosis or modify the fibrosis process.

The peptide hormone relaxin is known for its ability to inhibit short-term collagen production from tissues and cell culture models. Relaxin has shown to have anti-fibrotic effects in various in vitro and in vivo models. Unemori et al., J. Clin. Invest. 1996. 98:2739-2745 “Relaxin Induces an Extracellular Matrix-degrading Phenotype in Human Lung Fibroblasts In Vitro and Inhibits Lung Fibrosis in a Murine Model In Vivo,” state that relaxin inhibit lung fibrosis in a murine model. The importance of relaxin on inhibiting fibrosis is highlighted by the development of relaxin deficient mice (RLX-KO). See Samuel et al., FASEB J. (Nov. 1, 2002) 10.1096 “Relaxin deficiency in mice is associated with an age related progression of pulmonary fibrosis”; Samuel et al., Annals of the New York Academy of Sciences. Volume 1041, Relaxin and Related Peptides: Fourth International Conference, pages 173-181, May 2005. The Relaxin Gene-Knockout Mouse: A Model of Progressive Fibrosis. In those RLX-KO mice, from 6-9 months of age and onwards, all organs of RLX-KO mice, particularly male mice, underwent progressive increases in tissue weight and collagen content compared with wild-type animals. The increased fibrosis contributed to bronchiole epithelium thickening and alveolar congestion (lung), atrial hypertrophy and increased ventricular chamber stiffness (heart) in addition to glomerulosclerosis (kidney). Treatment of RLX-KO mice with recombinant human relaxin in early and developed stages of fibrosis caused the reversal of collagen deposition in the lung, heart, and kidneys.

The natural form of relaxin has a very short serum half life (less than 10 min) after intravenous administration. See Chen et al., Pharm Res. 1993 June; 10(6): 834-8. “The pharmacokinetics of recombinant human relaxin in nonpregnant women after intravenous, intravaginal, and intracervical administration.” Therefore, continuous infusion of relaxin is required to have therapeutic effects, which is inconvenient and costly, particular for chronic disease like fibrosis.

Idiopathic pulmonary fibrosis (IPF) is a chronic, progressive form of lung disease characterized by fibrosis of the supporting framework (interstitium) of the lungs. By definition, the term is used only when the cause of the pulmonary fibrosis is unknown. The median survival time is 2-5 years from the time of diagnosis. There is no satisfactory treatment or FDA approved treatment exists at present. Belomycin-induced lung fibrosis is the most commonly used animal model in studying lung fibrosis. See Coker et al., “Transforming growth factor β1 (TGF β1) and Endothelin-1 (ET-1) play important roles in fibrosis, particular in lung fibrosis” Eur Respir J 1998; 11: 1218-1221. Pulmonary fibrosis: cytokines in the balance. Park et al., Am. J. Respir. Crit. Care Med., Volume 156, Number 2, August 1997, 600-608, “Increased Endothelin-1 in Bleomycin-induced Pulmonary Fibrosis and the Effect of an Endothelin Receptor Antagonist”; Giaid et al., The Lancet, Volume 341, Issue 8860, Pages 1550-1554. Expression of endothelin-1 in lungs of patients with cryptogenic fibrosing alveolitis.” Both TGF β1 and ET-1 induce collagen production and extracellular matrix turn over; they also induce one another to form a positive feedback loop.

In the example below, long-lasting Relaxin-Fc fusion protein was investigated its effect on bleomycin-induced lung fibrosis in an in vivo mouse model and on TGF β1 induced ET-1 production in an in vitro cell-based model.

### Example 6

### Material and Methods

Animals. Studies were performed on 8-wk-old male C57BL/6J mice. They were allowed free access to water and commercial chow. All animal experiments were performed in accord with institutional guidelines set forth by the Institutional Animal Care and Usage Committee (IACUC).
Cells and Materials. Human lung fibroblasts cells (HLF) was obtained from ATCC, and maintained in DMEM media supplement with 10% fetal calf serum (FCS). All chemical reagents, including bleomycin, were purchased from Sigma Sigma-Aldrich (St. Louis, Mo. 63103). Bleomycin was dissolved in physiological saline just before each experiment. Relaxin (natural form) was a gift from Dr. Amento at Molecular Medicine Resarch Institute in Sunnyvale, Calif.

### TGF Beta 1 Induced Endothelin-1 (ET-1) Assay

At day 0, human lung fibroblasts cells (HLF, ATCC) were seeded in a 96-well collagen coated-plate (BD Biosciences) at cell density 40,000 cell/well and incubated overnight in 37° C., 5% CO2. At day 1, media was discarded from plate. 100 ul of serum-free media with the desired concentration of the natural form of relaxin, or relaxin-Fc, or media alone were added to the corresponding well, incubated for 1 hr in 37° C., 5% CO2. After 1 hour of incubation, TGF beta-1 in serum-free media was added to each well so the final TGF beta-1 concentration was 5 ng/ml. Following 24 hr incubation, 100 ul from each well was collected for Endothelin-1 (ET-1) measurement by Quntikine ELISA (R&D).

Animal Lung Fibrosis Induction and Experimental Design. Experiments were designed to examine the role of Relaxin-Fc in Bleomycin-induced fibrosis. According to previous reports, the collagen content of the lungs peaks 3 weeks after a single administration of bleomycin. See Lindenschmidt et al., Toxicol. Appl. Pharmacol. 85:69-77. “Intratracheal versus intravenous administration of bleomycin in mice: acute effects.”; Hesterberg et al., Toxicol. Appl. Pharmacol. 60:360-370, “Bleomycin-induced pulmonary fibrosis: correlation of biochemical, physiological, and histological changes.”

At day 0, animals were anesthetized by injection of ketamine intraperitoneally. A volume of 75 ul containing belomycin (1 unit/kg) or control saline was instilled through oropharyngeal aspiration. Animals were given Relaxin-Fc or Saline twice weekly at day 1, 4, 8, 10, 14, and 17 at a dose 4 ug/kg in 200 ul volume through tail vein or orbital vein injections. Animals were killed at Day 21. Serums were collected for PK confirmation. The right lungs were removed and fixed in formaldehyde for histology evaluation. The left lungs were collected and frozen at −80° C. and analyzed later for hydroxyproline content.

ELISA assay for Relaxin measurement and ET-1 measurement.

Relaxin measurement was performed using a Relaxin ELISA kit (Immundiagnostik AG). Endothelin-1 (ET-1) measurement was performed by using Quntikine ELISA kit (R&D).

Measurement of lung hydroxyproline. Collagen deposition was estimated by determining the total hydroxyproline content of the lung. As most collagen has been shown to contain 14% 4-HYP for various connective tissues, the 4-hydroxyproline would be a factor to estimate the collagen of biological specimen. Determination of 4-hydroxyproline was based on alkaline hydrolysis, oxidation with chloramine-T, formation of chromophere and measure absorbance at 560 nm (A560). The procedure of measuring 4-hydroxyproline is well established and described in G. Kesava Reddy and Chukuku Enwemeka, Clinical Biochem 1996, June V29:225 “A simplified method for the analysis of hydroxyproline in biological tissues.”

The amount of hydroxyproline in tissues was determined against a standard curve generated using known concentration of hydroxyproline (Sigma). Results were expressed as micrograms of hydroxyproline per lung.

Statistics. Data are expressed as means+/−SE unless otherwise stated. Statistical analyses were performed on the data through single-factor ANOVA among more than two groups and with Student's unpaired t-test for comparisons of two groups, all showing a P value of less than 0.05.

### Example 6

### Results

### Long-lasting Relaxin-Fc Inhibited TGF Beta 1 Induced ET-1 Production in HLF Cells.

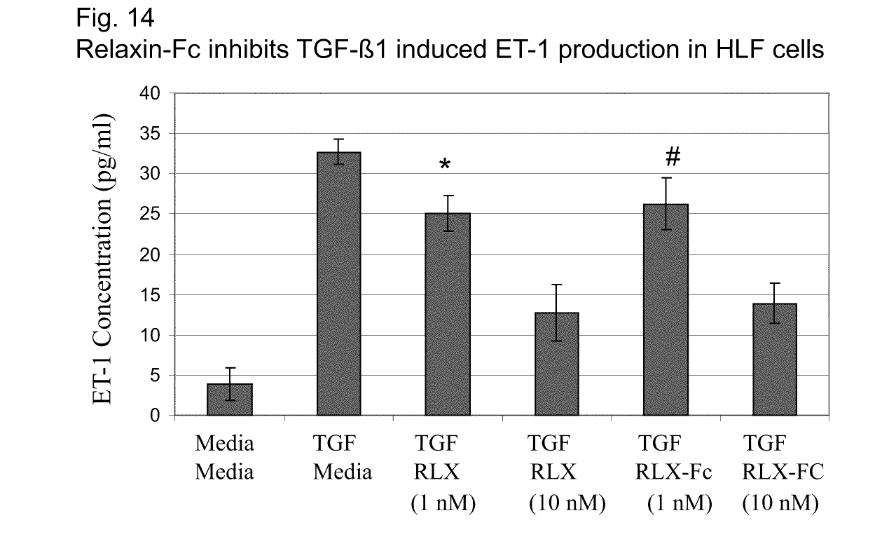

TGF beta can induce ET-1 production by HLF cells from control 3.9 pg/ml to 32.7 pg/ml. RLX can inhibit TGF induced ET-1 production in a dose dependent manner (23.3% at 1 nM, and 61.1% at 10 nM); while RLX-Fc demonstrated the comparable inhibition potency (19.7% at 1 nM, and 57.4% at 10 nM). See FIG. 14.

### Long-Lasting Relaxin-Fc Inhibited Bleomycin-Induced Fibrosis in a Murine Model.

Relaxin-Fc fusion protein was tested for its ability to inhibit bleomycin-induced pulmonary fibrosis in a mouse model. Bleomycin (1 U/kg) or saline was instilled through oropharyngeal aspiration at a volume of 75 ul at day 0. Relaxin-Fc (4 ug/kg) or vehicle (0.9% of saline) was administered by intravenous injection via the tail vein at day 1, 4, 7, 10, 14, and 17. Relaxin-Fc 4 ug/kg iv injection biweekly is appropriately equivalent area under curve (AUC) exposure in molarity level to nature form relaxin dose 140 ug/kg/days, administrated IV continuously. Circulating Relaxin-Fc levels were measured by ELISA in blood drawn at the termination of the experiments (data not shown). In each animal, relaxin levels approximated 15-20 ng/ml in both saline/Relaxin-Fc and bleomycin/Relaxin-Fc treatment groups and were undetectable in mice not receiving human relaxin.

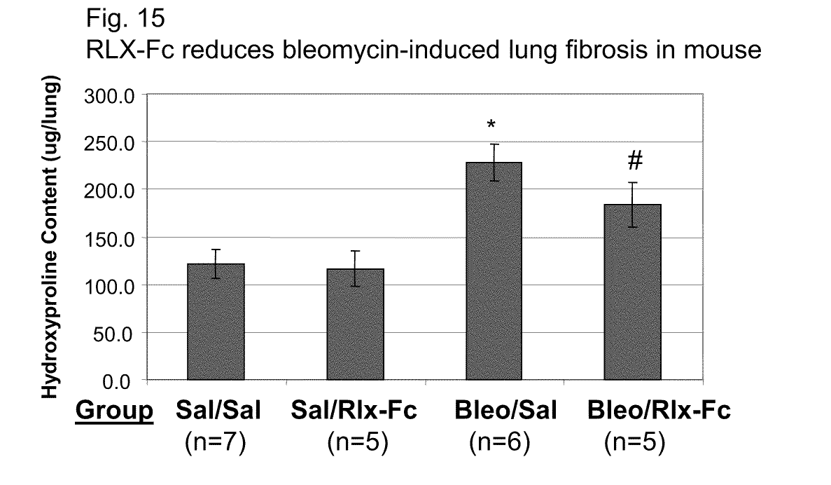

Compared to an un-induced group (Sal/Sal), the level of total hydroxyproline in lung in the bleomycin-induced group increased significantly (228.2+/−19.4 ug for bleomycin induced, and 121.8+/−15.1 ug for un-induced), meaning that bleomycin induced fibrosis in the lung (FIG. 15). While compared to bleomycin group, Relaxin-Fc treatment group of bleomycin-induced animals resulted in significant reduction in total hydroxyproline by 19.3% (FIG. 15).

### Example 6

### Conclusion

In this study, HLF cell-based in vitro system was used to test the inhibitory effect of RLX-Fc on TGF beta 1 induced ET-1 production. In this TGF beta 1 induced ET-1 system, TGF beta 1 is the inducer and ET-1 is the induced cytokine produced by HLF cells. The data demonstrated that RLX-Fc has an inhibitory effect on TGF-induced ET-1 production in an in vitro assay system. Although the mechanism and signal pathway of inhibition is not clear, it is known from this study that Rlx-Fc is able to block the TGF beta induced signal. ET-1 is also involved in pulmonary arterial hypertension (PAH), and ET-1 inhibitors (Tracleer, and Bosenta) have been approved by FDA to treat PAH. Therefore, RLX-Fc and related long acting forms of relaxin are expected to have therapeutic value in treating PAH patients.

In in vivo bleomycin-induced lung fibrosis animal model, RLX-Fc twice weekly administration resulted in significant allevation in lung fibrosis measured by total hydroxyproline. The data confirmed previous studies that continuous s.c administration of relaxin can reduce lung fibrosis in belomycin-induced lung fibrosis.

### Example 7

### Relaxin Increases Urine Flow Rate in Rats Indicating Enhanced Kidney Function

Female Sprague-Dawley rats, 5-6 wks old (body weight 130 g) were purchased from Charles River (Wilmington, Mass.). On day 0, the rats were treated with human Relaxin-Fc (RLX-Fc) at a dose of 8.0 ug/kg or vehicle control (PBS) via tail vein injection. On day B2 (baseline), Day 2, and Day 4, all rats were put into metabolic cages. The 24-hour urine volume was collected and measured. The urine flow rate was calculated using the following formula:

Urine flow rate=24-hour urine volume/1440minutes/body weight

The effect on urine flow of human relaxin-Fc administration to normal animals is shown in Table 1 below. Data was normalized against vehicle control group (PBS) on the day of sample collection. *P, <0.05 compared with PBS-treated control group. Treatment of Rlx-Fc (8.0 ug/kg) to normal rats enhanced urine flow rate at day 2 and 4 by 127% and 123% respectively.

| Treatment Groups | Day 2 | Day 4 |
|---|---|---|
| PBS Control | 1.0 | 1.0 |
| RLX-Fc 8 ug/kg | 1.27 ± 0.10 | 1.23 ± 0.04 |

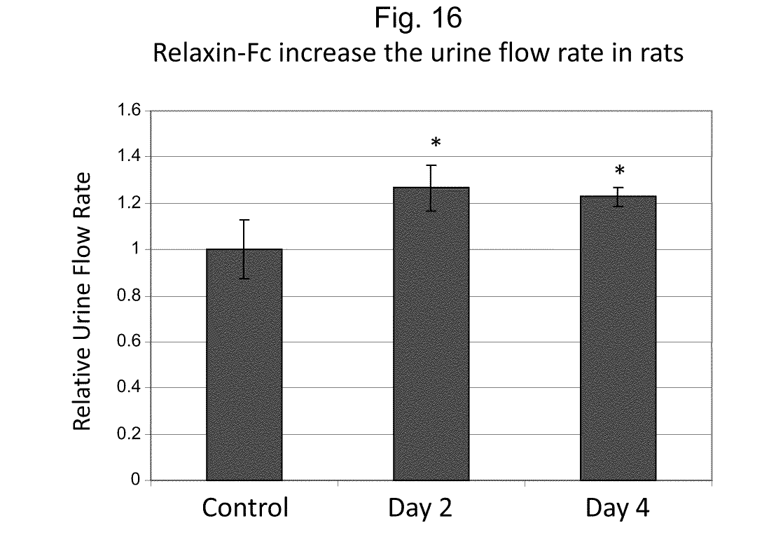

The results are also shown graphically in FIG. 16.

### Example 8

### Template-Based Computer Modeling to Predict RLX-(L)-Fc Fusion Protein Structure

### Introduction

Adding linker(s) between Relaxin and the Fc fragment may help the new fusion protein refold appropriately to a relaxin structure which binds to the appropriate receptors and maintains (or even improves) its desired function. Linkers are often composed of flexible residues like glycine and serine so that the adjacent protein domains are free to move relative to one another, but can also be other amino acid polymers (see e.g., U.S. Pat. No. 7,271,149) or other polymers (see US Application No. 2009/0181037 [Heavner]: listing as suitable polymers: polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and alpha-β-Poly-[(2-hydroxyethyl)-DL-aspartamide, and the like, or mixtures thereof, as well as a number of other compounds polymers and combinations, all of which are incorporated by reference). Longer linkers may be used when it is necessary to ensure that two adjacent domains do not sterically interfere with one another.

Commonly used linkers include but not limited to (Gly4Ser)n (n=or >1), (Ser-Gly-(Ser-Ser-Ser-Ser-Gly)2-Ser), (Gly-Gly-Ser-Gly)n (n=1-5), and (Ser-Gly-(Ser-Ser-Ser-Ser-Gly)2-Ser-) as they are known to be unstructured and flexible, so as to connect two proteins together while not interfering in the 3D structures of the each unit. The linking polymer is selected by structural modeling of the fusion protein and selection of a linking polymer such that the fusion protein has a predicted structure similar enough to the predicted structure of a relaxin-Fc fusion protein that it is predicted to have the same function as a relaxin-Fc fusion protein.

### Modeling Methods

Protein structure prediction aims to obtain 3D models of proteins by an optimized combination of experimental structure solution and computer-based structure prediction. It is a well-known and widely accepted technique that using structural genomics to predict protein structures was already in wide use several years ago [Burley S K, 1999, Nat Genet, 23:151-157; Chandonia J M, 2006, Science, 311:347-351]. Two factors will dictate the success of the structure prediction: experimental structure determination of optimally selected proteins and efficient computer modeling algorithms. Where similar structures are found in the Protein Data Bank (PDB) library, the protein structure prediction can be made using template-based modeling (TBM)—if not, one uses free modeling.

Since the structure of relaxin H2 and antibody Fc fragment can be obtained from PDB library by PSI-BLAST search, template-based modeling can be used to predict 3D structure of a Rlx-L-Fc fusion protein. Modeling is conducted in two steps: first, the known structure Relaxin H2 and Fc fragment are identified as templates, and the target sequences (Rlx-L-Fc) are aligned to the template structure. Second, structural frameworks are built by copying the aligned regions or by satisfying the spatial restraints from templates, and the unaligned loop regions and additional side-chain atoms are structured. The software(s) used for modeling are available either from commercial sources, e.g., CCP4 (available from CCP4 at Oxon, UK http://www.ccp4.ac.uk/) or free internet sources (such as PyMOL).

### Results and Conclusion

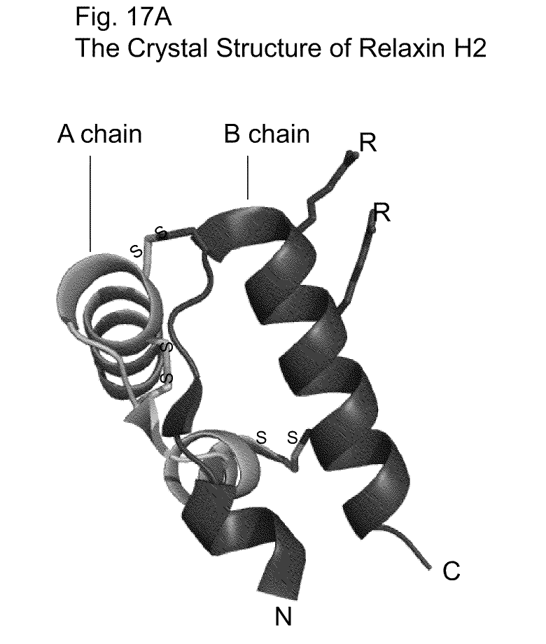

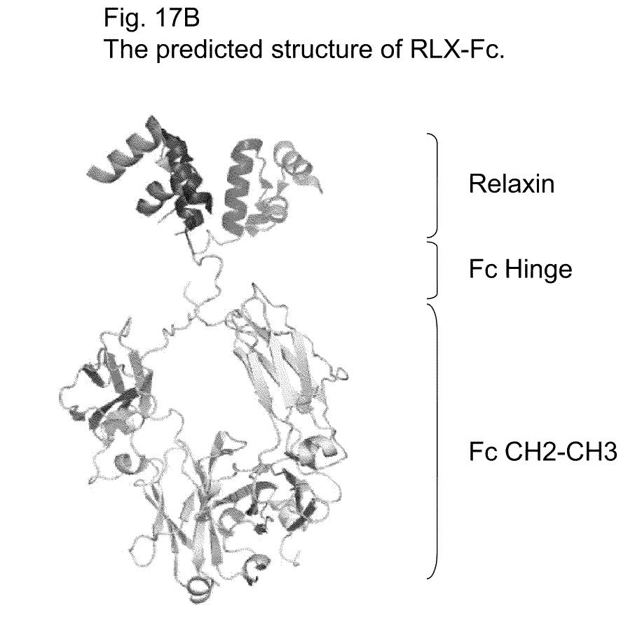

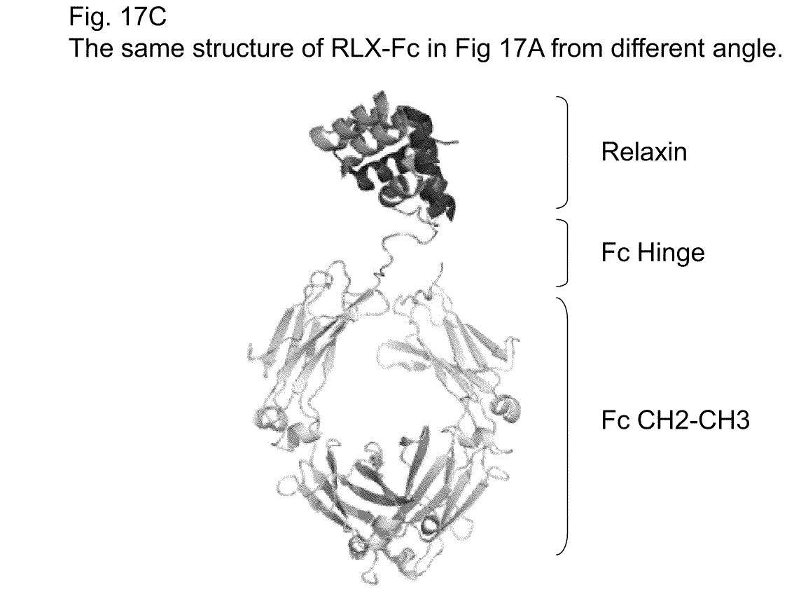

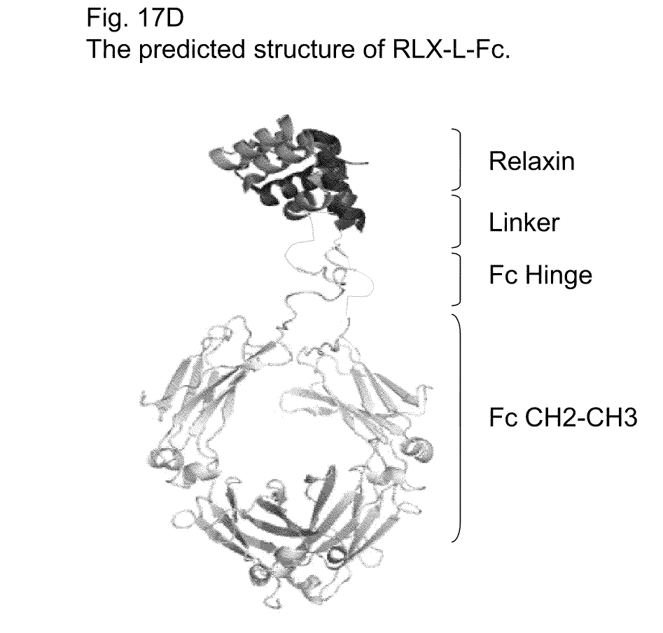

Full-length models on Rlx-L-Fc fusion protein are constructed by copying the template framework and by computer based structure modeling. Since the structures of relaxin H2 and Fc fragment are known, and the characteristics of the linkers are also well know, the modeling is relatively straightforward and the Rlx-L-Fc structure(s) can be predicted with high level of confidence. FIG. 17A is the crystal structure of native relaxin H2 which was obtained from PDB library. The relative positions of the A- and B-chains and the interconnecting cystine bridges are indicated. The arginines (R) of the B-chain, which are suggested to be involved in receptor binding, are also shown. FIG. 17B is the predicted structure of Rlx-Fc. The crystal structure of Fc fragment obtained from the PDB library is used as the template. Fc hinge (part of Fc fragment) which can be seen clearly between Rlx and Fc CH2 and CH3 gives Rlx space and flexibility to refold appropriately. FIG. 17 C is the same structure of Rlx-Fc in FIG. 17A from different angle. FIG. 17D is the predicted structure of Rlx-L-Fc, which includes the linker (Gly4Ser)₃. Fusion proteins with other linkers such as (Ser-Gly-(Ser-Ser-Ser-Ser-Gly)2-Ser), (Gly-Gly-Ser-Gly)n (n=1-5), and (Ser-Gly-(Ser-Ser-Ser-Ser-Gly)2-Ser-) were also modeled and showed the same structure as for the (Gly4Ser)3 linker (structures not shown). Other linkers in the fusion protein, or fusion proteins with other Fc portions (including mutants, truncated Fcs or variants) can be modeled the same way.

From the template-based modeling conducted here, it is predicted with a high degree of confidence that the RLX-L-Fc fusion protein(s) with different linkers have the same structure in the relaxin domain. It is also predicted that those RLX-L-Fc fusion protein(s) bind to the same RLX receptor(s) as native form relaxin does.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that only the following claims define the scope of the invention and that methods and structures within the scope of these claims and their equivalents be covered thereby.

What is claimed is:

1. A fusion protein comprising A and B chains of a human relaxin and at least a portion of a constant immunoglobulin domain such that said fusion protein, as compared to the corresponding human relaxin that lacks said constant immunoglobulin domain: (i) exhibits a longer serum half-life in vivo; and (ii) exhibits similar intracellular cAMP generation in cells treated with said fusion protein as compared to the same cell type treated with the corresponding human relaxin that lacks said constant immunoglobulin domain.

2. The fusion protein of claim 1, wherein said constant immunoglobulin domain is joined to said A chain or said B chain of said human relaxin.

3. The fusion protein of claim 2, comprising an additional linker amino acid sequence between said constant immunoglobulin domain and said A chain or said B chain to which it is joined.

4. The fusion protein of claim 1 wherein the cells are THP-1 cells or other cell lines or primary cells responding to Relaxin stimulation.

5. The fusion protein of claim 3 wherein the additional linker amino acid sequence has G and S in the proportion: (G4S)N, where N is 1 to X; (Ser-Gly-(Ser-Ser-Ser-Ser-Gly)2-Ser), (Gly-Gly-Ser-Gly)N where N is 1 to 5; or (Ser-Gly-(Ser-Ser-Ser-Ser-Gly)-Ser-).

6. The fusion protein of claim 3 wherein the additional linker amino acid sequence is SEQ ID No. 2.

7. The fusion protein of claim 1, comprising from N-terminus to C-terminus, said B chain, said A chain, and said constant immunoglobulin domain, but lacking a C chain of human relaxin.

8. The fusion protein of claim 1, comprising from N-terminus to C-terminus, said constant immunoglobulin domain, said B chain, and said A chain, but lacking a C chain of human relaxin.

9. The fusion protein of claim 1, comprising from N-terminus to C-terminus, said B chain, a C chain of a human relaxin, said A chain, and said constant immunoglobulin domain.

10. The fusion protein of claim 1, comprising from N-terminus to C-terminus, said constant immunoglobulin domain, said B chain, a C chain of a human relaxin, and said A chain.

11. The fusion protein of claim 1, wherein the constant immunoglobulin domain comprises an Fc region of a heavy chain IgG immunoglobulin.

12. The fusion protein of claim 1, wherein the constant immunoglobulin domain is modified such that its ADCC activity is lower than that of the corresponding unmodified constant immunoglobulin domain.

13. The fusion protein of claim 1, wherein the heavy chain IgG immunoglobulin is the γ4 chain.

14. The fusion protein of claim 1, wherein the constant immunoglobulin domain is modified such that it has an increased serum half-life compared to the corresponding unmodified constant immunoglobulin domain.

15. The fusion protein of claim 1 wherein the constant immunoglobulin domain has the amino acid sequence of SEQ ID No. 4, SEQ ID No. 5 or the mutant Fc sequence of SEQ ID No. 13.

16. The fusion protein of claim 1, wherein the human Relaxin is H2 Relaxin.

17. The fusion protein of claim 1, wherein said fusion protein competes with said human relaxin for binding of a human relaxin receptor.

18. The fusion protein of claim 17, wherein said human relaxin receptor is RXFP1, RXFP2, RXFP3, RXFP4, FSHR (LGR1), LHCGR (LGR2), TSHR (LGR3), LGR4, LGR5, LGR6, LGR7 (RXFP1), or LGR8 (RXFP2).

19. The fusion protein of claim 1 further including a tag to aid in affinity purification of the fusion protein.

20. The fusion protein of claim 19 wherein the tag is six histidines in succession.

21. The fusion protein of claim 19 wherein the tag is chitin binding protein (CBP), maltose binding protein (MBP), and glutathione-S-transferase (GST), Isopeptag, Histidine-tag, or HA-tag.

22. The fusion protein of claim 19 wherein the relaxin-tag fusion protein has the amino acid sequence shown in SEQ ID No. 6.

23. The fusion protein of claim 19 wherein the tag is inserted between the B chain and the A chain of the human relaxin portion of the fusion protein.

24. A pharmaceutical composition comprising the fusion protein of claim 1 and a pharmaceutically acceptable carrier.

25. A recombinant polynucleotide comprising a coding sequence that encodes the fusion protein of claim 1.

26. A recombinant polynucleotide of claim 25 having the sequence of SEQ ID No. 12 or SEQ ID No. 14.

27. A host cell incorporating the recombinant polynucleotides of claim 25.

28. A host cell incorporating the recombinant polynucleotides of claim 26.

29. A vector incorporating the recombinant polynucleotides of claim 25.

30. A vector incorporating the recombinant polynucleotides of claim 26.


SEQUENCE LISTING (16 sequences)

SEQ ID NO: 1 - DNA, 452 bp, Homo sapiens
atggccaggtacatgctgctgctgctcctggcggtatgggtgctgaccggggagctgtggccgggagctgaggcccgggcagcgccttacggggtcaggctttgcggccgagaattcatccgagcagtcatcttcacctgcgggggctcccggtggagacgatcagacatcctggcccacgaggctatgggagataccttcccggatgcagatgctgatgaagacagtctggcaggcgagctggatgaggccatggggtccagcgagtggctggccctgaccaagtcaccccaggccttttacagggggcgacccagctggcaaggaacccctggggttcttcggggcagccgagatgtcctggctggcctttccagcagctgctgcaagtgggggtgtagcaaaagtgaaatcagtagcctttgctagtttgagggctgggcagccgtggg

SEQ ID NO: 2 - protein, 16 aa, Artificial Sequence
  Description of Artificial Sequence Synthetic peptide
GGSGGSGGGGSGGGGS

SEQ ID NO: 3 - protein, 185 aa, Homo sapiens
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 4 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 229 aa, Artificial Sequence
  Description of Artificial Sequence Synthetic polypeptide
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 191 aa, Artificial Sequence
  Description of Artificial Sequence Synthetic polypeptide
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCHHHHHH

SEQ ID NO: 7 - DNA, 597 bp, Artificial Sequence
  Description of Artificial Sequence Synthetic polynucleotide
aagcttgccaccatgcctaggctgttttttttccatctgctgggtgtttgcctcctgcttaatcagttttctagagcggtggcagatagctggatggaggaagtgatcaagctgtgtggacgggaactggtgagagcccagattgccatctgtggaatgtcaacctggagcaaacggtccttgtcacaggaagatgcgcctcaaaccccacgcccagtagcagagatcgtgccgagtttcattaacaaagatacagagaccatcaatatgatgtctgaatttgtcgcaaatttgccccaagaacttaagctgacactgtcagaaatgcagccagcgctcccccagttgcagcagcatgtccccgtgcttaaagacagcagcctgctttttgaggagtttaaaaagctgattcgaaaccggcagtccgaggctgcggattccagccccagcgaactgaagtacctggggttggatacgcacagccggaaaaaacggcagctgtactcagcactggcgaacaagtgttgtcatgttggctgtacaaaacgatccctggcaagattctgtcatcaccaccaccatcattgataactcgag

SEQ ID NO: 8 - protein, 417 aa, Artificial Sequence
  Description of Artificial Sequence Synthetic polypeptide
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1275 bp, Artificial Sequence
  Description of Artificial Sequence Synthetic polynucleotide
aagcttgccaccatgccacggctgttcttcttccacttgctgggtgtgtgtctgctcctgaatcagttctcaagagcagtcgctgactcctggatggaggaggttatcaagctgtgtggacgcgaactggtgcgcgctcagatcgcgatatgcgggatgtccacatggtcaaaacgctctttgtctcaagaggatgctccacagacacccagaccagtggccgagattgtccccagctttataaacaaagacactgagaccataaacatgatgtccgaattcgtcgcaaatctgcctcaggagcttaagctcactctctctgagatgcaaccagcactgcctcagctgcagcagcacgtccctgtgctgaaggactccagcctgttgtttgaagaatttaaaaaacttattcgcaaccgccagtccgaggccgctgactcaagcccctcagagctgaagtacctgggactggacactcacagtcgcaaaaagcgacagctctactcagcgctcgctaataagtgttgtcatgtgggatgcacaaagcggtctctcgccagattctgcgaacctaagagctgcgacaaaacccacacctgtccaccctgtccagcccccgagctcctgggaggcccgagtgtgtttctttttcccccgaagcccaaagataccctcatgatctcaaggaccccagaggtgacatgcgtggtagtcgacgtgagccacgaagatcccgaggtgaagtttaactggtatgttgacggggtagaagttcacaatgctaagactaagccgcgcgaggaacagtataattccacgtatagggttgtctctgtcctgaccgtactgcatcaagactggctgaacggtaaggaatacaaatgcaaagtttccaataaagccctgcccgctccaattgagaaaacaatctctaaagcgaaggggcaaccgcgcgagccccaggtgtataccctgcctcccagtcgggacgaactcacaaagaaccaggtgagtctgacttgtctcgtcaaggggttctacccgtctgacattgccgtcgagtgggagagtaacggtcagcccgaaaacaactataagaccacgcctcccgttctcgattcagacggcagcttttttctgtacagcaagctgaccgtcgataaatctcgatggcagcaggggaatgtattttcatgttccgtgatgcacgaggccctccacaaccactacacacagaagtccctgagcctgtccccagggaaatgataactcgag

SEQ ID NO: 10 - protein, 414 aa, Artificial Sequence
  Description of Artificial Sequence Synthetic polypeptide
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 433 aa, Artificial Sequence
  Description of Artificial Sequence Synthetic polypeptide
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCGGSGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1323 bp, Artificial Sequence
  Description of Artificial Sequence Synthetic polynucleotide
aagcttgccaccatgccacggctgttcttcttccacttgctgggtgtgtgtctgctcctgaatcagttctcaagagcagtcgctgactcctggatggaggaggttatcaagctgtgtggacgcgaactggtgcgcgctcagatcgcgatatgcgggatgtccacatggtcaaaacgctctttgtctcaagaggatgctccacagacacccagaccagtggccgagattgtccccagctttataaacaaagacactgagaccataaacatgatgtccgaattcgtcgcaaatctgcctcaggagcttaagctcactctctctgagatgcaaccagcactgcctcagctgcagcagcacgtccctgtgctgaaggactccagcctgttgtttgaagaatttaaaaaacttattcgcaaccgccagtccgaggccgctgactcaagcccctcagagctgaagtacctgggactggacactcacagtcgcaaaaagcgacagctctactcagcgctcgctaataagtgttgtcatgtgggatgcacaaagcggtctctcgccagattctgcggcggcagtgggggctctggtgggggtggttccggcggagggggttcagaacctaagagctgcgacaaaacccacacctgtccaccctgtccagcccccgagctcctgggaggcccgagtgtgtttctttttcccccgaagcccaaagataccctcatgatctcaaggaccccagaggtgacatgcgtggtagtcgacgtgagccacgaagatcccgaggtgaagtttaactggtatgttgacggggtagaagttcacaatgctaagactaagccgcgcgaggaacagtataattccacgtatagggttgtctctgtcctgaccgtactgcatcaagactggctgaacggtaaggaatacaaatgcaaagtttccaataaagccctgcccgctccaattgagaaaacaatctctaaagcgaaggggcaaccgcgcgagccccaggtgtataccctgcctcccagtcgggacgaactcacaaagaaccaggtgagtctgacttgtctcgtcaaggggttctacccgtctgacattgccgtcgagtgggagagtaacggtcagcccgaaaacaactataagaccacgcctcccgttctcgattcagacggcagcttttttctgtacagcaagctgaccgtcgataaatctcgatggcagcaggggaatgtattttcatgttccgtgatgcacgaggccctccacaaccactacacacagaagtccctgagcctgtccccagggaaatgataactcgag

SEQ ID NO: 13 - protein, 433 aa, Artificial Sequence
  Description of Artificial Sequence Synthetic polypeptide
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCGGSGGSGGGGSGGGGSEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 14 - DNA, 1306 bp, Artificial Sequence
  Description of Artificial Sequence Synthetic polynucleotide
ggtaccggccggccaccatgccacggctgttcttcttccacttgctgggtgtgtgtctgctcctgaatcagttctcaagagcagtcgctgactcctggatggaggaggttatcaagctgtgtggacgcgaactggtgcgcgctcagatcgcgatatgcgggatgtccacatggtcaaaacgctctttgtctcaagaggatgctccacagacacccagaccagtggccgagattgtccccagctttataaacaaagacactgagaccataaacatgatgtccgaattcgtcgcaaatctgcctcaggagcttaagctccatcaccaccaccatcatactctctctgagatgcaaccagcactgcctcagctgcagcagcacgtccctgtgctgaaggactccagcctgttgtttgaagaatttaaaaaacttattcgcaaccgccagtccgaggccgctgactcaagcccctcagagctgaagtacctgggactggacactcacagtcgcaaaaagcgacagctctactcagcgctcgctaataagtgttgtcatgtgggatgcacaaagcggtctctcgccagattctgcgaacctaagagctgcgacaaaacccacacctgtccaccctgtccagcccccgagctcctgggaggcccgagtgtgtttctttttcccccgaagcccaaagataccctcatgatctcaaggaccccagaggtgacatgcgtggtagtcgacgtgagccacgaagatcccgaggtgaagtttaactggtatgttgacggggtagaagttcacaatgctaagactaagccgcgcgaggaacagtataattccacgtatagggttgtctctgtcctgaccgtactgcatcaagactggctgaacggtaaggaatacaaatgcaaagtttccaataaagccctgcccgctccaattgagaaaacaatctctaaagcgaaggggcaaccgcgcgagccccaggtgtataccctgcctcccagtcgggacgaactcacaaagaaccaggtgagtctgacttgtctcgtcaaggggttctacccgtctgacattgccgtcgagtgggagagtaacggtcagcccgaaaacaactataagaccacgcctcccgttctcgattcagacggcagcttttttctgtacagcaagctgaccgtcgataaatctcgatggcagcaggggaatgtattttcatgttccgtgatgcacgaggccctccacaaccactacacacagaagtccctgagcctgtccccagggaaatgataacctgcaggctcgag

SEQ ID NO: 15 - protein, 430 aa, Artificial Sequence
  Description of Artificial Sequence Synthetic polypeptide
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCGGSGGSGGGGSGGGGSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 6 aa, Artificial Sequence
  Description of Artificial Sequence Synthetic 6xHis tag
HHHHHH